(19) United States
(12) Patent Application Publication
(10) Pub. No.: US 2008/0194518 A1
(43) Pub. Date: Aug. 14, 2008

(54) Antimicrobial Compositions

(76) Inventors: Pradip MOOKERJEE, Flemington, NJ (US); Shira Kramer, Timonium, MD (US); Alexander Josowitz, Balitmore, MD (US); April Zambelli-Weiner, Hampstead, MD (US)

Correspondence Address: MILES & STOCKBRIDGE PC, 1751 PINNACLE DRIVE, SUITE 500, MCLEAN, VA, 22102-3833, US

(21) Appl. No.: 11/964,153

(22) Filed: Dec. 26, 2007

Related U.S. Application Data
(63) Continuation-in-part of application No. 11/644,900, filed on Dec. 26, 2006.
(60) Provisional application No. 60/753,175, filed on Dec. 23, 2005.

(51) Int. Cl. A61K 31/01; A61K 31/722; A61K 31/724; A61K 31/70; A61K 31/194; A61P 31/04; A01P 1/00; A61K 31/14; A61K 31/05; A61K 31/66; A61K 31/13

(52) U.S. Cl. 514/55; 514/161; 514/579; 514/58; 514/762; 514/108; 514/23; 514/731; 514/574; 514/642

(57) ABSTRACT

An antimicrobial composition, including a synergistic combination of three or more agents as an active ingredient. Each of the three or more potentiating agents can be selected from the following types of compounds: sequestering agents, carbohydrates and carbohydrate derivatives, terpenes/terpenoids, amines and amine derivatives, plant-derived oils, sulfonates, phenols, fatty acids, dibenzofuran derivatives, organo isothiocyanates, quaternary ammonium compounds, peroxides and peroxide donors, and macrolide polyenes. At least two of the three or more potentiating agents are not of the same type of compound. The antimicrobial composition can have strong antimicrobial efficacy in control of microorganisms having resistance to currently used antimicrobials.

### CROSS REFERENCE

This application claims the benefit of U.S. Provisional Application No. 60/753,175, filed Dec. 23, 2005, and U.S. application Ser. No. 11/644,900 filed Dec. 26, 2006 the entire contents of each being hereby incorporated by reference in its respective entirety.

### FIELD OF THE INVENTION

This invention relates to antimicrobial compositions.

### BACKGROUND

Pathogenic microorganisms, including, for example, bacteria, viruses, and fungi, are responsible for a host of human diseases, ranging from more minor ailments, such as upper and lower respiratory tract infections, to potentially fatal infections, such as listeriosis.

During the last 100 years, major progress has been made in combating diseases caused by pathogenic microorganisms with the development of copious pharmaceutical and non-pharmaceutical agents to be used in treatments. For example, in pharmacy, an antibiotic agent can be used to treat bacterial infections within humans, whereas a chemical-based agent can be used for external treatment (e.g., on a hard surface) to prevent contamination and transmission to humans, as in the case of Listeria in ready-to-eat meat and poultry processing plants.

While agents have been developed that are generally effective against various pathogens, there is increasing evidence that the use of such agents has certain limitations which warrant concern. Specifically, certain strains of pathogenic microorganisms have become increasingly resistant to one or more antimicrobials, thereby rendering the standard courses of treatment ineffective. Accordingly, higher doses of antimicrobial treatments can be required to achieve efficacy, which can result in undesirable side effects and toxicity, both human and environmental. In addition, many antimicrobial treatments are not designed to combat biofilm, which is a major contributor to antimicrobial resistance development, both biologically (in vivo) and environmentally.

### BRIEF DESCRIPTION OF THE TABLES

TABLE 1 provides example proposed embodiments.

TABLE 2 provides example synergistic combinations—MIC data against MRSA

TABLE 3 provides example synergistic combinations—MIC data against E. coli.

TABLE 4 provides further synergy data.

TABLE 5 provides comparative data for selected examples.

### SUMMARY OF THE INVENTION

In an aspect, the invention features an antimicrobial composition. The antimicrobial composition includes a synergistic combination of three or more agents, such agents can be antimicrobial potentiating agents or can be antimicrobial agents. Each of the three or more agents is independently selected from varying compounds. The agent can be selected from the following groups: sequestering agents, carbohydrates and carbohydrate derivatives, terpenes/terpenoids, amines and amine derivatives, plant-derived oils, sulfonates, phenols, fatty acids, dibenzofuran derivatives, organo isothiocyanates, quaternary ammonium compounds, peroxides and peroxide donors, and macrolide polyenes. At least two of the three or more agents are not from the same group.

In another aspect, amine and amine derivatives can be further classified as amines, amine oxides, peptides, alkaloids, and dyes that have an amine functional group.

In another aspect, carbohydrates and carbohydrate derivatives can be further classified as carbohydrates and fatty acid polyol esters.

In another aspect, the invention features a method for treating a microbial infection. The method includes administering the present antimicrobial composition as an active ingredient. Proposed methods of administration include but are not limited to parenteral, oral, sublingual, transdermal, topical, intranasal, aerosol, intraocular, intratracheal, intrarectal, vaginal, gene gun, dermal patch, eye drop, ear drop and mouthwash.

In another aspect, the invention features a method for producing a pharmaceutical composition. The method includes mixing the present antimicrobial composition with a pharmaceutically acceptable excipient.

In another aspect, the invention features a method of treating wounds to prevent and treat infections. The method includes administering the present antimicrobial composition as an active ingredient alone or in combination with an antibiotic.

In another aspect, the invention features a method of treating oral infections. The method includes administering the present antimicrobial composition as an active ingredient alone or in combination with an antibiotic.

In another aspect, the invention features a method for treating a microorganism-contaminated surface. The method includes applying to the surface the present antimicrobial composition.

In another aspect, the invention features a method of sterilizing medical devices and equipment. The method includes applying the present antimicrobial composition to the device or equipment.

In another aspect, the invention features a method of preserving substances including but not limited to food and beverage products, cosmetics, personal care products, household products, paints, and wood. The method includes administering the present antimicrobial composition as an active ingredient.

In another aspect, the invention features a method of formulating a nutriceutical or cosmeceutical. The method includes administering the present antimicrobial composition as an active ingredient alone or in combination with a nutriceutical or cosmeceutical.

In another aspect, the invention features a method of preventing the formation of bacterial biofilms and provides a method of treating bacterial biofilms on surfaces as well as in the human body. The method includes administering the present antimicrobial composition as an active ingredient alone or in combination with an antimicrobial or antibiotic.

In another aspect, the invention features a method of impregnating materials with a bactericidal and bacteriostatic ingredient. The method includes impregnating surfaces with the present antimicrobial composition.

One or more of the following features can also be included.

The antimicrobial composition can include, as an active ingredient, an antibacterial agent, an antifungal agent, or an antiviral agent. The antimicrobial composition can include a pharmaceutically acceptable excipient.

Microbial infections to be treated by the antimicrobial composition can include bacterial infections caused by drug-resistant bacteria. Likewise, microorganisms of the microorganism-contaminated surfaces to be treated by the antimicrobial composition can include drug-resistant microorganisms.

Embodiments of the invention can have one or more of the following advantages.

The antimicrobial compositions of the present invention can have strong antimicrobial efficacy in the control of microorganisms having resistance to currently used antimicrobials.

In accordance with the present invention, an antimicrobial potentiating agent need not be an antimicrobial agent itself, and can synergistically boost the efficacy of other agents in the antimicrobial composition by, for example, impairing another function(s) in a cell that is essential for cell viability. Such potentiating agents can include compounds that individually have shown poor antimicrobial activity in screening tests. The antimicrobial compositions can employ (i) potentiating agents alone as active antimicrobial compounds, (ii) a potentiating agent(s) with an antimicrobial compound(s) to actively reverse the resistance of microorganisms to the antimicrobial compound(s) and make the antimicrobial compound(s) effective, or (iii) a potentiating agent(s) with an antimicrobial compound(s) as an effective combination against non-resistant microorganisms.

Using the compositions of the present invention, a microorganism can be treated in the absence of a known antimicrobial agent, using an antimicrobial agent in lower concentrations, or using an antimicrobial agent which is not effective when used in the absence of the potentiating agent(s). Thus, methods of treatment using the antimicrobial compositions can be useful as substitutes for treatments using an antimicrobial agent alone at high dosage levels (which can cause undesirable side effects), or as treatments for which there is a lack of a clinically effective antimicrobial agent. The methods of treatment can be especially useful for treatments involving microorganisms that are susceptible to particular antimicrobial agents as a way to reduce the dosage of those particular agents. This can reduce the risk of side effects, and it can also reduce the selection effect for highly resistant microorganisms resulting from consistent high level use of a particular antimicrobial agent.

Further aspects, features, and advantages will become apparent from the following.

### DESCRIPTION OF AN EMBODIMENT

The term “antimicrobial” as used herein refers to the ability of an agent or composition to beneficially control or kill pathogenic, spoilage, or otherwise harmful microorganisms, including, but not limited to, bacteria, fungi, viruses, protozoa, yeasts, mold, and mildew.

The term “potentiating agent” as used herein refers to any compound that can enhance the efficacy of an antimicrobial composition as a whole by interacting with microorganisms in a way that facilitates or enhances the antimicrobial characteristics of the composition.

The term “active ingredient” as used herein refers to the combination of potentiating agents and, optionally, antimicrobial agents that are responsible for the antimicrobial characteristics of the antimicrobial composition.

The term “synergistic” as used herein refers to the interaction of two or more agents so that their combined effect is greater than the sum of their individual effects.

In an embodiment, an antimicrobial composition can include a combination of three or more potentiating agents as an active ingredient.

For example, at least one of the three or more potentiating agents of the antimicrobial composition can be a sequestering agent. Preferred examples of sequestering agents include, but are not limited to, quinolines, phosphorus acid derivatives, carboxylate sequestrants, natural protein sequestrants, and cyclodextric sequestrants. Particularly preferred examples of sequestering agents include 8-hydroxyquinoline, ethylenediaminetetraacetic acid (EDTA), 1-hydroxyethylidene-1,1-diphosphonic acid (HEDP), sodium pyrophosphate, potassium hypophosphite, sodium tripolyphosphate, salicylic acid, 2-hydroxypropyl-a-cyclodextrin, hypophosphorous acid, citric acid, and lactoferrin.

At least one of the three or more potentiating agents of the antimicrobial composition can be a carbohydrate or carbohydrate derivative. Preferred examples of carbohydrates or carbohydrate derivatives include, but are not limited to, polyol ethers and esters. Particularly preferred examples of carbohydrates or carbohydrate derivatives include, but are not limited to, polysaccharides, oligosaccharides and fatty acid polyol esters. Particularly preferred examples of carbohydrates include 2-hydroxypropyl-α-cyclodextrin, chitosan, octyl glucoside, and glycerol monocaprylate.

At least one of the three or more potentiating agents of the antimicrobial composition can be a terpene/terpenoid. Preferred terpene/terpenoids contain at least two isoprenoid substructural units. Particularly preferred examples of terpenes/terpenoids include, but are not limited to, linalool, limonene, nerolidol, totarol, and ursolic acid

At least one of the three or more potentiating agents of the antimicrobial composition can be an amine or amine derivative. Examples of amines or amine derivatives include, but are not limited to, amines, peptides, alkaloids, dyes with an amine functional group, amine oxides and quaternary ammonium compounds. Preferred examples of amines or amine derivatives include, but are not limited to, nisin, piperine, methylene blue, N,N-bis-(3-aminopropyl)dodecylamine, cetylpyridinium chloride, lauryl dimethylamine oxide, and sodium pyrithione.

At least one of the three or more potentiating agents of the antimicrobial composition can be a quaternary ammonium compound. Preferred examples of quaternary ammonium compounds include, but are not limited to L-carnitine and ADBACs (alkyl dimethyl benzyl ammonium chloride).

At least one of the three or more potentiating agents of the antimicrobial composition can be a plant-derived oil. Preferred examples of plant-derived oils include, but are not limited to, allyl isothiocyanate and carvacrol.

At least one of the three or more potentiating agents of the antimicrobial composition can be a sulfonate. Preferred examples of sulfonates include, but are not limited to, naphthalene sulfonic acid and sodium lignosulfonate.

At least one of the three or more potentiating agents of the antimicrobial composition can be a phenol. Preferred examples of phenols include one or more phenolic functional groups. Particularly preferred examples of phenols include, but are not limited to, salicylic acid, tannic acid, carvacrol, activin, octyl gallate, thymol and catechins.

At least one of the three or more potentiating agents of the antimicrobial composition can be a fatty acid. A preferred example of a fatty acid includes, but is not limited to, phospholipid CDM.

At least one of the three or more potentiating agents of the antimicrobial composition can be a dibenzofuran derivative. A preferred example of a dibenzofuran derivative includes, but is not limited to, usnic acid.

At least one of the three or more potentiating agents of the antimicrobial composition can be an organo isothiocyanate. A preferred example of an organo isothiocyanate includes, but is not limited to, allyl isothiocyanate.

At least one of the three or more potentiating agents of the antimicrobial composition can be a peroxide or peroxide donor. Preferred examples of peroxides/peroxide donors include, but are not limited to, hydrogen peroxide and sodium carbonate peroxyhydrate.

At least one of the three or more potentiating agents of the antimicrobial composition can be a macrolide polyene. A preferred example of a macrolide polyene includes, but is not limited to, natamycin.

Preferred combinations of potentiating agents as active ingredients are chosen on the basis of natural or near-natural origin as well as safety profile.

As stated above, potentiating agents of the antimicrobial composition need not be an antimicrobial agent itself. Indeed, in certain embodiments, each of the three or more potentiating agents is not, on its own, an antimicrobial agent. Thus, certain embodiments advantageously kill or inhibit the growth of microorganisms via antimicrobial activity that is not otherwise observed for any of the individual components alone.

Preferred embodiments of this invention are antimicrobial combinations comprised of individual agents that, in combination, can be used at concentrations significantly lower (generally, but not limited to 40-95%) than required individually to achieve antimicrobial efficacy, antibiotic synergy, or resistance reversal. The ability to use very low concentrations of individual agents in combination to achieve high-level antimicrobial efficacy or antibiotic synergy is a primary advantage of this invention.

For example, the synergistic combination of an amine derivative, a carbohydrate derivative, and a sequestrant required individual components at a concentration level 5% of what would have been required individually to achieve the same level of antimicrobial efficacy.

In addition, embodiments in which none of the three or more potentiating agents is, on its own, an antimicrobial agent can be used in combination with an antimicrobial agent to enhance the efficacy of the antimicrobial agent. In these embodiments, the three or more potentiating agents can be used to enhance the efficacy of an antimicrobial agent against, for example, a resistant strain of microorganism. More generally, antimicrobial compositions combining one or more antimicrobial agents and three or more potentiating agents advantageously can be able to kill or inhibit the growth of microorganisms at lower concentrations of the one or more antimicrobial agents.

For example, in some embodiments of antimicrobial compositions, three or more potentiating agents, none of which, on its own, is an antimicrobial agent, can be combined with an antimicrobial agent, such as, for example, an antibacterial agent, an antifungal agent, and an antiviral agent.

Preferred examples of antibacterial agents that can be combined with three or more potentiating agents include, but are not limited to, beta-lactams, aminoglycosides, glycopeptides, fluoroquinolones, macrolides, tetracyclines, and sulphonamides. Preferred examples of beta-lactams include, but are not limited to, penicillins, cephalosporins, carbapenems, and monobactams.

Other beta-lactams that can be included in the antimicrobial compositions include, but are not limited to, imipenem, meropenem, saneftrinem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriazone, cefurozime, cefuzonam, cephaaceterile, cephalexin, cephaloglycin, cephaloridine, cephalothin, cephapirin, cephradine, cefmetazole, cefoxitin, cefotetan, azthreonam, carumonam, flomoxef, moxalactam, amidinocillin, amoxicillin, amicillin, azlocillin, carbenicillin, benzylpenicillin, carfecillin, cloxacillin, dicloxacillin, methicliloin, mezlocillin, nafcillin, oxacillin, penicillin G, piperacillin, sulbenicillin, temocillin, ticarcillin, cefditoren, cefdinir, ceftibuten, and Cefozopran.

Macrolides that can be included in the antimicrobial compositions include, but are not limited to, azithromycin, clarithromycin, erythromycin, oleandomycin, rokitamycin, rosaramicin, roxithromycin, troleandomycin, telithromycin and other ketolides.

Quinolones that can be included in the antimicrobial compositions include, but are not limited to, amifloxacin, cinoxacin, ciprofloxacin, enoxacin, fleroxacin, flumequine, loMefloxacin, nalidixic acid, norfloxacin, ofloxacin, levofloxacin, oxolinic acid, pefloxacin, difloxacin, marbofloxacin, rosoxacin, temafloxacin, tosufloxacin, sparfloxacin, clinafloxacin, trovafloxacin, alatrofloxacin, grepafloxacin, moxifloxacin, gatifloxacin, gemifloxacin, nadifloxacin, and rufloxacin.

Tetracyclines that can be included in the antimicrobial compositions include, but are not limited to, chlortetracycline, demeclocyline, doxycycline, lymecycline, methacycline, minocycline, oxytetracycline, and tetracycline.

Aminoglycosides that can be included in the antimicrobial compositions include, but are not limited to, amikacin, arbekacin, butirosin, dibekacin, fortimicins, gentamicin, kanamycin, netilmicin, ribostanycin, sisomicin, spectinomycin, streptomycin, tobramycin, clindamycin, and lincomycin.

Other oxazolidinones that can be included in the antimicrobial compositions include, but are not limited to, linezolid and eperezolid.

Preferred examples of antifungal agents that can be combined with three or more potentiating agents, include, but are not limited to, triazoles, imidazoles, polyene antimycotics, allylamines, echinocandins, cerulenin, and griseofulvin.

Preferred examples of antiviral agents that can be combined with three or more potentiating agents, include, but are not limited to, reverse transcriptase inhibitors, nucleoside reverse transcriptase inhibitors (NRTIs), nucleoside analog reverse transcriptase inhibitors (NARTIs), guanine analogs, protease inhibitors, neuraminidase inhibitors, and nucleoside antimetabolites. Other examples of antiviral agents which can be combined with three or more potentiating agents, include, but are not limited to, acyclovir, ribavarine, zidovudine, and idoxuridine.

In some embodiments, one or more of the three or more potentiating agents is, on its own, an antimicrobial agent. For example, nisin is a peptide (amine derivative) and is mentioned above as an example potentiating agent to be used in the antimicrobial compositions. Nisin is also known to have, on its own, antimicrobial activity.

Particularly striking is the ability of embodiments of the antimicrobial compositions to extend the range of antimicrobial effectiveness against microorganisms previously considered to have limited effectiveness against one or more of the antimicrobial compounds of the antimicrobial compositions. For example, antibiotic activities of polymyxins have been considered to be restricted to gram-negative bacteria, such as E. coli and Pseudomonas aeruginosa. However, embodiments of the antimicrobial compositions extend the antimicrobial effect of polymyxins to gram-positive bacteria such as Staphylococcus aureas, and to fungi, including yeasts such as Candida albicans.

In some embodiments, an antimicrobial composition can include a combination of three or more agents as an active ingredient. Each of the three or more agents can be independently selected from the following different types of compounds: sequestering agents, carbohydrates and carbohydrate derivatives, terpenes/terpenoids, amines and amine derivatives, plant-derived oils, sulfonates, phenols, fatty acids, dibenzofuran derivatives, organo isothiocyanates, quaternary ammonium compounds, peroxides and peroxide donors, and macrolide polyenes; and antimicrobial agents.

Additional examples of antimicrobial agents that can be combined with other agents in the antimicrobial compositions include, but are not limited to, anti-tuberculosis drugs, antileprosy drugs, oxazolidelones, bisdiguanides, quaternary ammonium compounds, carbanilides, salicyanilides, hydroxydiphenyls, organometallic antiseptics, halogen antiseptics, peroxygens, amine derivatives, terpenes, terpenoids, phenols, alkaloids, natural alkyl isothiocyanates, organic sulfonates, fatty acid esters, and alkyl glycosides. Other examples of antimicrobial agents which can be combined with other agents in the antimicrobial compositions include, but are not limited to, hydantoins, 3-iodo-2-propynyl-butyl-carbamate (IPBC), isothiazolones, benzisothiazolones (BIT), chlorhexidine, 2,2-dibromo-3-nitrilo propionamide (DBNP), 2-bromo-2-nitropropane-1,3-diol, ureas, nisin, pyrithiones, N,N-bis(3-aminopropyl)dodecylamine, lauryl amine oxide, and cetylpyridinium chloride (CPC). Still other examples of antimicrobial agents which can be combined with other agents in the antimicrobial compositions include, but are not limited to, didecyldimethylammonium chloride, cetyl trimethyl ammonium bromide, benzethonium chloride, methylbenzethonium chloride, hydroxydiphenyls such as dichlorophene and tetrachlorophene; organometallic and halogen antiseptics such as zinc pyrithione, silver sulfadiazine, silver uracil, and iodine; peroxygens such as hydrogen peroxide, sodium perborate, persulfates, and peracids; and amine derivatives.

In certain embodiments, at least two of the three or more agents are not of the same type of compound. For example, in a proposed antimicrobial composition, the synergistic combination of three or more potentiating agents includes two amines or amine derivatives and a sequestering agent. In an alternative example, the synergistic combination of three or more potentiating agents includes nisin, piperine, and 8-hydroxyquinoline.

In another proposed antimicrobial composition, the synergistic combination of three or more potentiating agents includes a terpene/terpenoid, a plant-derived oil, and a sequestering agent. In an alternative example, the synergistic combination of three or more potentiating agents includes nerolidol, allyl isothiocyanate, and 1-hydroxyethylidene-1,1-diphosphonic acid (HEDP).

In yet another proposed antimicrobial composition, the synergistic combination of three or more potentiating agents includes a terpene/terpenoid, a dibenzofuran derivative, and a sequestering agent. In an alternative example, the synergistic combination of three or more potentiating agents comprises nerolidol, usnic acid, and 2-hydroxypropyl-α-cyclodextrin.

In still another proposed antimicrobial composition, the synergistic combination of three or more agents includes a terpene/terpenoid, an amine or amine derivative, and a sequestering agent. In an alternative example, the synergistic combination of three or more agents includes limonene, sodium pyrithione, and salicylic acid.

In an additional proposed antimicrobial composition, the synergistic combination of three or more agents includes an amine or amine derivative, a quaternary ammonium compound, and a sequestering agent. In an alternative example, the synergistic combination of three or more agents includes lauryl amine oxide, cetylpyridinium chloride (CPC), and potassium ethylenediaminetetraacetic acid.

In an additional proposed antimicrobial composition, the synergistic combination of three or more agents includes an amine or amine derivative, a carbohydrate or carbohydrate derivative, and a sequestering agent. In an alternative example, the synergistic combination of three or more agents includes piperine, chitosan, and 1-hydroxyethylidene-1,1-diphosphonic acid (HEDP).

In an additional proposed antimicrobial composition, the synergistic combination of three or more agents includes terpene/terpenoid, an amine or amine derivative, and a sequestering agent. In an alternative example, the synergistic combination of three or more agents includes nerolidol, N,N-bis-(3-aminopropyl)dodecylamine, and salicylic acid.

In an additional proposed antimicrobial composition, the synergistic combination of three or more agents includes two terpene/terpenoids, and a sequestering agent. In an alternative example, the synergistic combination of three or more agents includes nerolidol, limonene, and 1-hydroxyethylidene-1,1-diphosphonic acid (HEDP).

In an additional proposed antimicrobial composition, the synergistic combination of three or more agents includes a terpene/terpenoid, a carbohydrate or carbohydrate derivative, and a sequestering agent. In an alternative example, the synergistic combination of three or more agents includes nerolidol, octyl glucoside, and salicylic acid.

In an additional proposed antimicrobial composition, the synergistic combination of three or more agents includes a terpene/terpenoid, a phenol, and a sequestering agent. In an alternative example, the synergistic combination of three or more agents includes nerolidol, thymol, and HEDP.

For certain embodiments of the antimicrobial compositions, the agents can be selected for use based on a multi-modal combination strategy. Without being bound to any theory, it is believed that combinations of agents can have non-receptor-mediated modes of action and can effect breakdown of microbial cells via multiple modes of action, including cell rupture. Consequently, the combinations can be less likely to induce the type of rapid resistance frequently observed with actives that have receptor-mediated modes of action. Embodiments of the antimicrobial compositions can also advantageously avoid certain toxicological problems, particularly allergic responses, often associated with the therapeutic use of novel proteins.

For these embodiments, the modes of action can generally be described as involving physical undermining of cell structure, instead of interception of biochemical pathways used by most other antimicrobials such as antibiotics. Antimicrobial compositions having an active ingredient(s) designed to have non-receptor-mediated modes of action can be less likely to engender resistance development through natural selection and gene transfer. For example, a potentiating agent(s) can synergistically boost the efficacy of the composition as a whole by impairing some other function(s) in the cell that is essential for cell viability through mechanisms such as, for example, essential metal sequestration, multi-drug resistance (MDR) pump inhibition, cell membrane permeabilization, and inhibition of repair mechanisms that are activated when cell membranes are disrupted. For example, without being bound to any theory, sequestering agents can restrict the availability of metal ions that are needed to repair damage to cytoplasmic membranes of cells that result from the action of some antimicrobial active ingredients. As another example, nerolidol can have lytic activity that provides improved access of antimicrobial active ingredients to other intracellular targets.

As one example, antimicrobial compositions containing agents selected for use based on a multi-modal combination strategy can include: (1) sequestering agents; (2) efflux pump inhibiting compounds; and (3) cell membrane disrupter compounds. With respect to sequestering agents, for instance, the efficacy and resilience to adverse effects of antimicrobial resistance can be overcome by a mechanism that combines chelation of iron by siderophores with cell membrane disruption. An efflux pump inhibitor is a compound which specifically interferes with the ability of an efflux pump to export its normal substrate, or other compounds such as an antimicrobial. An efflux pump refers to a protein assembly which exports substrate molecules from the cytoplasm or periplasm of a cell, in an energy-dependent fashion.

Example cell membrane disrupters that can be included in the antimicrobial compositions include, but are not limited to, nerolidol, berberine HCl, lysozyme, oil of oregano, nisin, phospholipid CDM, tea tree oil, lactoperoxidase, curcumin, maltol, caffeic acid, and sodium lignosulfonate. Example efflux pump inhibitors that can be included in the antimicrobial compositions include, but are not limited to, green tea extract, quinine, cremaphor EL, capsaicin, PEG (400) dioleate, pluronic F127, and 5,5-dimethylhydantoin. Example sequestering agents, in addition to those described earlier herein above, that can be included in the antimicrobial compositions include, but are not limited to, salicylhydroxamic acid, lactoferrin, 8-hydroxyquinoline SO₄, Na₂EDTA, Na₄pyrophosphate, desferrioxamine mes, pyrithione, and ferritin.

In general, the sequestering agents that can be included in embodiments of the antimicrobial compositions can be compounds having a Fe⁺³ complex with a stability constant greater than 10²⁰. The following more fully describes some of the above listed compounds that can be included in the antimicrobial compositions.

The primary constituents of oil of oregano (Origanum vulgare) are Carvacrol and Thymol. The sum of these two constituents can range from 50% to 90% of the oil. Other common constituents include beta-bisabolene, p-cymene, and a number of further monoterpenoids (e.g., 1,8-cineol, gamma-terpinene, terpinene-4-ol and terpinene-4-yl acetate) in amounts between, for example, 1% and 5%.

Tea tree oil (Meleleuca alternifolia) can contain at least 30% terpinen-4-ol, 10 to 28% gamma-terpinene, 5 to 13% alpha-terpinene and can contain up to 15% 1,8-cineole and up to 12% p-cymene.

Green tea extract (Camellia sinensis) can contain 60 to 90% total polyphenols and 30 to 55% (−)-epigallocatechin gallate.

Phospholipid CDM is a 37% aqueous solution of sodium coco PG-dimonium chloride phosphate.

Cremophor EL is an ethoxylated castor oil (CAS Number: 61791-12-6).

Pluronic F127 is an ethylene oxide/propylene oxide block copolymer terminating in primary hydroxyl groups.

Tomadol 91-2.5 is a mixture of ethoxylated fatty alcohols consisting of C9 to C11 alcohols with an average of 2.5 moles of ethylene oxide per molecule.

Capsaicin, which can be included in embodiments of the antimicrobial compositions, can function as an efflux pump inhibitor and can contribute to reversal of antimicrobial resistance. Capsaicin is known to have TRPV1 activity (transient receptor potential vanilloid 1), wherein the receptor is a ligand-gated ion channel, activated by agonists such as capsaicin. The following non-limiting list of naturally occurring chemicals, which can be included in embodiments of the antimicrobial compositions, have structural similarities with capsaicin, and are known or believed to also show similar TRPV1 activity and provide for reversal of antimicrobial resistance: 6,7-dihydrocapsaicin, nordihydrocapsaicin, homocapsaicin, nordihydrocapsaicin, capsiate, 6,7-dihydrocapsiate, nordihydrocapsiate, zingerone, [3-6]-, [8]-, [10]-, and [12]-gingerol, [3-6]-, [8]-, [10]-, and [12]-shogaol, zingibroside R-1, piperine, paradol, dehydroparadol, resiniferatoxin, olvanil, arvanil, linvanil, and anandamide.

The following non-limiting list of naturally occurring 1,4-dialdehydes, which can be included in embodiments of the antimicrobial compositions, show similar TRPV1 activity to capsaicin. Naturally occurring 1,4-dialdehydes with TRPV1 activity: (+) and (−)-isovelleral, (+) and (−)-isoisovelleral, aframodial, cinnamodial, desacetylscalaradial, polygodial, isocopalendial, scalaradial, warburganal, ancistrodial, B-acaridial, merulidial, and scutigeral.

The following are related terpenoids with TRPV1 activity, which can be included in embodiments of the antimicrobial compositions: cinnamosmolide; cinnamolide; drimenol; and hebelomic acid F.

The following is a non-limiting list of synthetic capsaicin analogs that can be substituted for naturally occurring capsaicin and that can be included in embodiments of the antimicrobial compositions. Synthetic capsaicin analogs: N-vanillyl octanamide; N-vanillyl nonanamide; N-vanillyl paaiperic acidamide; N-vanillyl decanamide; and N-vanillyl undecanamide.

The following is a non-limiting list of synthetic TRPV1 antagonists that can be included in embodiments of the antimicrobial compositions: N-[4-(nethylsulfonyl amino)benzyl]thiourea analogs; N-(4-chlorobenzyl)-N′-(4-hydroxy-3-iodo-5-methoxybenzyl)thiourea[IBTU]; isoquinolin-5-yl-ureas and -amides; 4-(2-pyridyl)piperazine-1-carboxamides; and 7-hydroxynaphthalen-1-yl-ureas and -amides.

Caffeic acid is a cell membrane disrupter and can provide for reversal of antimicrobial resistance. The following is a non-limiting list of naturally occurring compounds, which can be included in embodiments of the antimicrobial compositions, and that have structural similarity with caffeic acid and can provide for reversal of antimicrobial resistance: ferulic acid; isoferulic acid; o-coumaric acid; trans-p-coumaric acid; chlorogenic acid; cis & trans cinnamic acid; dihydrocinnamic acid; rosmarinic acid; lithospermic acid; carnosic acid; carnosolic acid; 3,4-dimethoxycinnamic acid; and 4-hydroxybenzoic acid.

In addition to the esters previously mentioned herein above, the following esters can also be included in embodiments of the antimicrobial compositions and can provide for reversal of antimicrobial resistance: methyl esters; phenethyl esters; 3-methylbut-2-enyl esters; and 3-methylbutyl esters.

Embodiments of the antimicrobial compositions can contain any of the components stated thus far herein, including salts, hydrates, polymorphs, and pseudopolymorphs thereof.

Embodiments of the antimicrobial compositions can contain acids, such as, for example, hydrochloric, hydrobromic, hydroiodic, sulphuric, sulfamic, sulfonic, phosphoric, acetic, lactic, succinic, oxalic, maleic, fumaric, malic, tartaric, citric, ascorbic, gluconic, benzoic, cinnamic, methanesulfonic and p-toluenesulfonic acid.

Embodiments of the antimicrobial compositions can contain cationic salts, such as, for example, those of alkali metals, such as, for example, lithium, sodium, or potassium, those of alkaline earth metals, such as, for example, magnesium or calcium, ammonium or organic amines such as, for example, diethanolamine and N-methylglucamine, guanidine or heterocyclic amines, such as, for example, choline, N-methyl-4-hydroxypiperi-dine, hydroxyethylpyrrolidine, hydroxyethylpiperidine, morpholine, hydroxyethylmorpholine, piperazine, N-methyl piperazine and the like, or basic amino acids such as, for example, optically pure or racemic isomers of arginine, lysine, histidine, tryptophan and the like.

Embodiments of the antimicrobial compositions can also include one or more of phenoxyethanol, tetrahydrofurfuryl alcohol (THFA), block copolymers based on ethylene oxide and propylene oxide, polyethylene glycol, and water.

Embodiments of the antimicrobial compositions can be used in methods for treating in vivo infections, promoting health in animals, especially mammals, by killing or inhibiting the growth of harmful microorganisms, disinfecting surfaces, and protecting materials from the harmful effects of microbial contaminants. For example, in some embodiments, the antimicrobial compositions can be used in methods for disinfecting surfaces and materials, including, but not limited to, bandages, bodily appliances, catheters, surgical instruments, and patient examination tables. In other embodiments, the antimicrobial compositions can be used in methods for combating resistant microorganisms through the ability to penetrate and remove biofilms.

Methods for treating microbial infections using embodiments of the antimicrobial compositions include, but are not limited to, oral treatments, parenteral administration, and topical application of an effective amount of the antimicrobial composition. The methods include methods for treating infections in humans and animals, especially mammals, caused by sensitive and resistant microbial strains using the antimicrobial compositions, wherein the active ingredient(s) increases the susceptibility of the microorganism to the antimicrobial agent. The methods also include methods for prophylactic treatment of a human or an animal, especially a mammal, including administering to the human or animal at risk of a microbial infection the antimicrobial compositions, wherein the active ingredient(s) decreases the pathogenicity of a microorganism in the human or animal.

In certain embodiments, the methods include contacting a bacterium or fungus with the potentiating agents in the presence of a concentration of antibacterial or antifungal agent below the minimum inhibitory concentration (MIC) of the antibacterial or antifungal agent for that bacterium or fungus.

In embodiments for treating in vivo infections, the antimicrobial compositions can be administered as an active ingredient either internally or externally. For external administration, the compositions can be used to treat, for example, infections of the skin or mucosal surfaces, corneas, infected cuts, burns, or abrasions, bacterial skin infections, or fungal infections (e.g., athlete's foot). For internal administration, the antimicrobial compositions can be useful for treating, for example, systemic bacterial infections, especially Staphylococcus infections. Antimicrobial compositions can also be administered internally by topical administration to mucosal surfaces, such as, for example, vaginal mucosa, for treatment of infections, particularly yeast infection.

In preferred embodiments, microbial infections to be treated can be due to bacteria, including, but not limited to, Streptococcus pneumoniae, Pseudomonas aeruginosa, Escherischia coli and Staphylococcus aureus. Indeed, embodiments of the antimicrobial compositions can be effective in controlling both Gram-positive and Gram-negative bacteria. In particularly preferred embodiments, microbial infections to be treated can be due to drug-resistant bacteria, including, but not limited to, resistant E. coli and methicillin-resistant Staphylococcus aureus (MRSA).

In embodiments of the methods for treatment, a pharmaceutically effective amount of the antimicrobial composition can be administered. A pharmaceutically effective amount means an amount of the active ingredient(s), i.e., the potentiating agents and, optionally, antimicrobial agent(s), which has a therapeutic effect. This can refer to the inhibition, to some extent, of the normal activities of microbial cells causing or contributing to a microbial infection. A therapeutically effective dose can also refer to that amount of the active ingredient(s) that results in amelioration of symptoms or a prolongation of survival in a patient, and can include elimination of a microbial infection. The doses of the potentiating agents and, optionally, antimicrobial agent(s), which are useful in combination as a treatment are therapeutically effective amounts. Thus, as used herein, a therapeutically effective amount means those amounts of potentiating agents and, optionally, antimicrobial agent(s), which, when used in combination, produce the desired therapeutic effect as judged by clinical trial results and/or model animal infection studies.

In certain embodiments, the potentiating agents and, optionally, antimicrobial agent(s) are combined in pre-determined proportions, and thus a therapeutically effective amount would be an amount of the combination. This amount, and the amount of the potentiating agents and, optionally, antimicrobial agent(s) individually, can be routinely determined, and will vary, depending on several factors, such as, for example, the particular microbial strain involved and the particular potentiating agents and, optionally, antimicrobial agent(s) used. This amount can further depend upon the patient's height, weight, sex, age and medical history. For prophylactic treatments, a therapeutically effective amount is that amount that would be effective if a microbial infection existed.

For embodiments of methods for treating, the therapeutically effective dose can be estimated initially from cell culture assays. For example, a dose can be formulated in animal models to achieve a circulating plasma concentration range that includes the IC₅₀ as determined in cell culture. Such information can be used to more accurately determine a useful dosage in a human.

The exact formulation, route of administration and dosage can be chosen by an individual physician in view of a patient's condition. Further, the dose and in some cases dose frequency can vary according to the age, body weight and response of the individual patient.

Embodiments of the antimicrobial compositions can include a pharmaceutically acceptable excipient. Excipients are substances that can mix with active ingredients to provide formulations. The main functions of excipients are to facilitate the manufacture, storage, and use of formulations. Excipients can also be said to facilitate and optimize the transfer of active ingredients to an intended target. Excipients that can be used in the antimicrobial compositions include, but are not limited to, fillers, extenders, emollients, wetting agents, lubricants, surfactants, solvents, diluents, carriers, binders, disintegrants, viscosity modifiers, preservatives, stabilizers, adhesives, film-forming agents, deodorants, hydrotropes, humectants, flavoring agents, coloring agents, and fragrances. For practical use, the active ingredients can be mixed with an excipient(s) to obtain an end-use formulation.

Due to the synergistic nature of the active ingredients, the antimicrobial compositions can be developed using decreased concentrations of active ingredients. The concentration of each ingredient shall be in a range as is generally known by one of ordinary skill in the art.

In certain embodiments, the antimicrobial compositions can contain as little as 0.39 ppm, or 0.000039 percent, of each active ingredient of the combination. The balance of the composition, if any, can be supplied in some embodiments by a suitable excipient(s). In these embodiments, an antimicrobial agent, if included, can be employed in a quantity less than that of the potentiating agents. In some embodiments of antimicrobial compositions, one hundred percent (100%) of the composition can be potentiating agents. Concentrations of potentiating agents and antimicrobial agents, if any, in use dilutions can range from 0.01 μg/ml to 10,000 μg/ml, the remainder of the use dilution preferably being excipients or diluents, such as, for example, water.

The antimicrobial compositions can be made using conventional procedures. For example, in some embodiments, components of the antimicrobial compositions can be conveniently dissolved or dispersed in an inert fluid medium that serves as an excipient. The term “inert” means that the excipient does not have a deleterious effect on the active ingredient(s) upon storage, nor does it substantially diminish its activity, nor does it adversely react with any other component of the composition.

Embodiments of antimicrobial compositions for in vivo administration can be provided as, for example, solutions, especially aqueous solutions, but they can alternatively be alcoholic solutions to increase the solubility of hydrophobic components. Such solutions can be especially convenient for oral administration, and can also be formulated for parenteral administration. For oral administration, ethanol can be preferred because of its low toxicity. Usually ethanol will be present in the minimum concentration needed to keep the components in solution. For external topical application, isopropanol can be used. Other formulations for oral administration can include, for example, solid dosage forms, such as, for example, tablets or capsules. Embodiments of antimicrobial compositions preferred for topical administration can be provided as, for example, emulsions, creams, or liposome dispersions, or as an ointment in a hydrophobic carrier, such as, for example, petrolatum.

Embodiments of the antimicrobial compositions can also be of other formulations. For example, a quantity of potentiating agents can be combined with a quantity of an antimicrobial agent(s), if any, in a mixture, e.g., in a solution or powder mixture. In such mixtures, the relative quantities of the potentiating agents and the antimicrobial agent(s), if any, can be varied as appropriate for the specific combination and expected treatment. In another example, the potentiating agents and the antimicrobial agent(s), if any, can be covalently linked in such manner that the linked molecules can be cleaved within the cell.

Other possibilities also exist, including, for example, serial administration of individual potentiating agents and the antimicrobial agent(s), if any. For example, in certain embodiments, the antimicrobial compositions can be constituted at the point of use, or alternatively two or more components of the compositions can be previously combined, in appropriate ratios, so that the antimicrobial compositions can be constituted at the point of use by adding the remaining components and acceptable carriers or modifying agents in appropriate ratios to achieve effective concentrations of composition components.

In embodiments of the methods for treating, the active ingredient(s) can be administered in pro-drug forms, i.e., the active compound(s) is administered in a form which is modified within the cell to produce the functional form.

Depending on the specific microorganism being treated, embodiments of the antimicrobial compositions can be formulated and administered systemically or locally. Suitable routes can include, for example, oral, rectal, transdermal, vaginal, transmucosal, or intestinal administration; parenteral delivery, including, but not limited to, intramuscular, subcutaneous, intramedullary, injections, as well as intrathecal, direct intraventricular, intravenous, intraperitoneal, intranasal, or intraocular injections. Dosage forms include, but are not limited to, solutions, suspensions, tablets, pills, powders, troches, dispersions, emulsions, capsules, injectable preparations, patches, ointments, creams, lotions, shampoos, dusting powders and the like.

Embodiments of pharmaceutical compositions suitable for oral administration can be presented as discrete units such as, for example, capsules, cachets, or tablets, or aerosol sprays, each containing a predetermined amount of the active ingredient(s), as a powder or granules, or as a solution or a suspension in an aqueous liquid, a non-aqueous liquid, an oil-in-water emulsion, or a water-in-oil liquid emulsion. Such compositions can be prepared by any of the methods of pharmacy, but all methods include the step of bringing into association the active ingredient with the excipient, which constitutes one or more ingredients. Embodiments of the pharmaceutical compositions can be prepared by uniformly and intimately admixing the active ingredient with liquid excipients or finely divided solid excipients or both, and then, if necessary, shaping the product into the desired presentation.

Embodiments of the antimicrobial compositions include, but are not limited to, compositions such as, for example, microemulsions, suspensions, solutions, elixirs, aerosols, and solid dosage forms. Excipients can be used in any case, and especially the case of oral solid preparations (such as, for example, powders, capsules and tablets), with the oral solid preparations being used in certain preferred embodiments. Particularly preferred oral solid preparations can be tablets.

Because of their ease of administration, tablets and capsules can represent in some embodiments the most advantageous oral dosage unit form, in which case solid pharmaceutical carriers can be preferably employed. For these embodiments, examples of suitable excipients include, but are not limited to, lactose, white sugar, sodium chloride, glucose solution, urea, starch, calcium carbonate, kaolin, crystalline cellulose and silicic acid, binders such as water, ethanol, propanol, simple syrup, glucose, starch solution, gelatine solution, carboxymethyl cellulose, shellac, methyl cellulose, potassium phosphate and polyvinyl pyrrolidone, disintegrants such as dried starch, sodium alginate, agar powder, laminaria powder, sodium hydrogen carbonate, calcium carbonate, Tween (fatty acid ester of polyoxyethylenesorbitan), sodium lauryl sulfate, stearic acid monoglyceride, starch, and lactose, disintegration inhibitors such as white sugar, stearic acid glyceryl ester, cacao butter and hydrogenated oils, absorption promoters such as quaternary ammonium bases and sodium lauryl sulfate, humectants such as glycerol and starch, absorbents such as starch, lactose, kaolin, bentonite and colloidal silicic acid, and lubricants such as purified talc, stearic acid salts, boric acid powder, polyethylene glycol and solid polyethylene glycol.

In certain embodiments, the tablet, if used, can be coated, and made into sugar-coated tablets, gelatine-coated tablets, enteric-coated tablets, film-coated tablets, or tablets containing two or more layers. If desired, tablets can be coated by standard aqueous or nonaqueous techniques.

In molding embodiments of the pharmaceutical compositions into pills, a wide variety of conventional excipients can be used. Examples include, but are not limited to, glucose, lactose, starch, cacao butter, hardened vegetable oils, kaolin and talc, binders such as gum arabic powder, tragacanth powder, gelatin, and ethanol, and disintegrants such as, for example, laminaria and agar.

In molding embodiments of the pharmaceutical compositions into a suppository form, a wide variety of conventional excipients can be used. Examples include, but are not limited to, polyethylene glycol, cacao butter, higher alcohols, gelatin, and semi-synthetic glycerides.

Other embodiments of the pharmaceutical compositions can be administered by controlled release means.

Embodiments of the pharmaceutical composition formulated into an injectable preparation can be formulated into a solution or suspension. Any conventional excipient can be used. Examples include, but are not limited to, water, ethyl alcohol, polypropylene glycol, ethoxylated isostearyl alcohol, polyoxyethylene sorbitol, and sorbitan esters. Sodium chloride, glucose or glycerol can also be incorporated into a therapeutic agent.

Embodiments of the antimicrobial compositions can contain, for example, ordinary dissolving aids, buffers, pain-alleviating agents, and preservatives, and optionally coloring agents, perfumes, flavors, sweeteners, and other drugs.

For topical application embodiments, there can be employed, as non-sprayable forms, viscous to semi-solid or solid forms comprising a carrier compatible with topical application and having a dynamic viscosity preferably greater than water. Formulations of these embodiments include, but are not limited to, solutions, suspensions, emulsions, creams, ointments, powders, liniments, salves, aerosols, etc., which can be, if desired, sterilized or mixed with auxiliary agents, e.g., preservatives, antioxidants, stabilizers, wetting agents, buffers or salts for influencing osmotic pressure, etc. For other topical application embodiments, sprayable aerosol preparations can be used wherein, for example, the active ingredient can be in combination with a solid or liquid inert carrier material.

For embodiments to be used in the disinfection of nonliving surfaces, such as, for example, countertops, surgical instruments, and bandages, antimicrobial compositions can be, for example, solutions, either aqueous or organic. For embodiments in which direct human contact with the disinfectant can be limited, such as, for example, in the disinfection of work surfaces or restrooms, mixed organic solutions can be appropriate, e.g., ethanol or isopropanol in water. Preferred alcohols for solvent purposes include, but are not limited to, ethyl, n-propyl, isopropyl, n-butyl, isobutyl, and t-butyl alcohols. Concentration of the alcohol in a mixed solvent system can range from 5% to nearly 100%. In these embodiments, there can be a cosolvent, such as, for example, be water or an aqueous buffer. In a majority of embodiments, the alcohol component can be limited to an amount necessary to keep the antibiotic and potentiator in solution.

A wide variety of applications are envisioned for the antimicrobial compositions, including, but not limited to, nutriceuticals to enhance health, preservatives to inhibit or prevent growth of microorganisms during manufacturing and in finished products, preservatives to inhibit or prevent growth of microorganisms in food and beverage products, stand-alone antimicrobials for direct food contact (e.g., produce wash), cosmeceuticals for promotion of skin health care, hard surface sanitation and disinfection, application to carcasses for the control of microorganisms, environmental remediation (e.g., mold and mildew), antibiotic synergism (resistance reversal), stand-alone antimicrobials for human and animal health care (topical, injectable, oral, pulmonary delivery), and decontamination of infectious biowarfare agents.

Example embodiments are illustrated in Table 1. Example synergistic combinations—MIC data against MRSA are illustrated in Table 2. Example synergistic combinations—MIC data against E. coli are illustrated in Table 3. Example synergy data are illustrated in Table 4. Comparative data for selected examples are illustrated in Table 5.

### EXAMPLES

Samples were prepared at a 1% w/w (10,000 ppm) concentration in their respective solvents.

### Example 1

### Preparation of Sample ST1-72-1

A small beaker was filled with approximately 80.0 ml of deionized water. 1.0 g of potassium ethylenediaminetetraacetic acid was added and dissolved. 1.0 g of cetylpyridinium chloride was added to the solution and dissolved. 1.0 g of Barlox 12 (lauryl amine oxide) was added using a pipette and dissolved. The solution was brought up to a total weight of 100.0 g with deionized water.

### Example 2

### Preparation of Sample ST1-73-1

A small beaker was filled with approximately 80.0 ml of phenoxyethanol. 1.0 g of nisin was added and dissolved. 1.0 g of piperine was added to the solution using and dissolved. 1.0 g of 8-hydroxyquinoline was added and dissolved. The solution was brought up to a total weight of 100.0 g with phenoxyethanol.

### Example 3

### Preparation of Sample ST1-76-3

A small beaker was filled with approximately 80.0 ml of phenoxyethanol. 1.0 g of 1-hydroxyethylidene-1,1-diphosphonic acid (HEDP) was added and dissolved. 1.0 g of nerolidol and 1.0 g of allyl isothiocynate was added to the solution using a pipette and dissolved. The solution was brought up to a total weight of 100.0 g with phenoxyethanol.

### Example 4

### Preparation of Sample ST1-78-1

A small beaker was filled with approximately 80.0 ml of phenoxyethanol. Using a pipette, 1.0 g of nerolidol was added. 1.0 g of 2-hydroxypropyl-α-cyclodextrin was added to the solution and dissolved. 1.0 g of usnic acid was added and dissolved. The solution was brought up to a total weight of 100.0 g with phenoxyethanol.

### Example 5

### Preparation of Sample ST2-8-2

A small beaker was filled with approximately 80.0 ml of phenoxyethanol. 1.0 g of sodium pyrithione was added and dissolved. 1.0 g of salicylic acid was then added to the solution. 1.0 g of limonene was added using a pipette. The solution was brought up to a total weight of 100.0 g with phenoxyethanol.

MIC testing and synergistic effects studies. Microorganisms were strains supplied by the ATCC. Staphylococcus aureus is a clinically significant member of the gram-positive group of bacterial pathogens. It gives rise to serious infections, and can produce bacteremia, endocarditis, and meningitis. Methicillin-resistant strains of Staphylococcus aureus (MRSA) were chosen for evaluation because they are a significant medical problem, particularly in view of the fact that methicillin is a drug of choice for treatment of S. aureus infection in the common penicillin-resistant strains. Escherichia coli was also chosen for evaluation. E. coli is a gram-negative pathogenic enterobacteriaceae, and is commonly used as a model organism for bacteria in general. The E. coli strain O157:H7, one of hundreds of strains of the bacterium E. coli, causes illness in humans.

Table 2 shows embodiments of novel antimicrobial and synergistic combinations. Good antimicrobial activity against MRSA is evidenced by a low Minimum Inhibitory Concentration (MIC), i.e., MIC<100, and synergy is evidenced by a synergy index (SI)<1.0. All individual components are present in the combination at a starting point of 10,000 μg/mL, or 1%. Combinations are serially diluted to obtain the MIC. Table 3 also shows embodiments of novel antimicrobial and synergistic combinations. Good antimicrobial activity against E. coli is evidenced by a low Minimum Inhibitory Concentration (MIC), i.e., MIC<100, and synergy is evidenced by a synergy index (SI)<1.0. All individual components are present in the combination at a starting point of 10,000 μg/mL, or 1%. Combinations are serially diluted to obtain the MIC.

Abbreviations in Tables 2 and 3 are as follows. MIC is the Minimum Inhibitory Concentration. MRSA is Methicillin-resistant Staphylococcus Aureus. E. coli is Escherichia coli. SI is the Synergy Index, wherein when the SI<1.0, there is synergy, a SI of 1.0 equals additivity, and a SI>1.0 equals antagonism. Pluronic F127 is Pluronic F127 microemulsion.

The Minimum Inhibitory Concentrations (MIC) were determined using tube dilution sensitivities. Dilutions of the combinations were added to bacterial growth media (tryptic soy broth) to result in a set of tubes with a concentration range of 0.1 mg/L-1000 mg/L. Overnight bacterial cultures were then added to these dilutions to produce a final concentration of 105 CFU/ml. The cultures were incubated overnight at 37° C. and MICs were recorded. The MIC was determined as the lowest concentration of a combination which prevented visible microorganism growth (e.g., turbidity). A culture growth control without compound and several culture sensitive reference agents were used as positive controls. The assays were performed in triplicate.

Synergistic effects of the antimicrobial compositions were also evaluated and the results reported in Tables 2 and 3. Dilutions of the compositions were added to bacterial growth media (tryptic soy broth) to result in a set of tubes with a concentration range of 0.1 mg/L-1000 mg/L of the combination. Overnight bacterial cultures were then added to this supplemented media to produce a final concentration of 105 CFU/ml.

Synergy is mathematically demonstrated by the industry accepted method described by S. C. Kull et al. in Allied Microbiology, Vol. 9, pages 538-541 (1961). As applied to this invention, it is as follows: Q_{A} is the ppm (MIC) of active substance A alone which produces an endpoint. Q_{B} is the ppm (MIC) of active substance B alone which produces an endpoint. Q_{C} is the ppm (MIC) of active substance C alone which produces an endpoint. Qₐ is the ppm (MIC) of active substance A, in the combination, which produces an endpoint. Q_{b} is the ppm (MIC) of active substance B, in the combination, which produces an endpoint. Q_{c} is the ppm (MIC) of active substance C, in the combination, which produces an endpoint. And so on for Qₙ components.

If the SI Of Qₐ/Q_{A}+Q_{b}/Q_{B}+Q_{c}/Q_{C} is less than one, synergy is indicated. A value greater than one indicates antagonism. A value equal to one indicates additivity. For example, for Sample ST1-73-1, Q_{A} is 1001 ppm, Q_{B} is 1001 ppm, Q_{C} is 1.56 ppm, Qₐ is 0.39 ppm, Q_{b} is 0.39 ppm, and Q_{c} is 0.39 ppm. Thus, the SI value for Sample ST1-73-1 is (0.39/1001)+(0.39/1001)+(0.39/1.56), or 0.251.

Preferred embodiments include ST1-72-1, ST1-73-1, ST1-76-3, ST1-78-1, ST2-8-2. These combinations are characterized by very low MICs and low synergy indices.

Additional synergy testing. Synergistic effects of antimicrobial compositions, further containing an antibiotic reference compound, were also evaluated. Specifically, four antibiotics representing different structural classes of antibiotics were tested: gentamicin (aminoglycoside), tetracycline, doxycycline (a member of the tetracycline family), and ciprofloxacin (fluoroquinolone), and the results are shown in Table 4. The particular combinations of potentiating agents identified by sample number in Table 4 are added in serial amounts with 0.5× the MIC of the antibiotic (i.e., a sub-effective concentration). The MIC presented in Table 4 under each antibiotic is the concentration of the combination of potentiating agents that was able to restore antimicrobial efficacy to 0.5×MIC of the antibiotic. The limit of the test in Table 4 is 0.1 μg/ml.

Dilutions of the combinations were added to bacterial growth media (tryptic soy broth) to result in a set of tubes with a concentration range of 0.1 mg/L-50 mg/L of the combination of potentiating agents plus ½ MIC of the antibiotic. Overnight bacterial cultures were then added to this supplemented media to produce a final concentration of 105 CFU/ml.

Because the antibiotic is present at ½ of its MIC, the MIC determined for the combination should be its usual value, if the effects of the two compounds are merely additive; greater than ½, if the compounds are antagonistic; and less than its usual value if the compounds are synergistic. The “Synergy Index” (SI) shown in Table 3 is the ratio of the MIC for the combination of potentiating agents in the presence of ½ MIC of the antibiotic to the MIC for the combination of potentiating agents alone. Similar to above, a SI value of less than 1.0 is indicative of synergy, a SI value of 1.0 indicates additivity, and an SI value greater than 1.0 is indicative of antagonism.

Sample ST2-8-2 demonstrates sufficient activity against resistant E. coli. In the presence of a sub-effective level of the antibiotic (i.e., ½ the MIC), ST2-8-2 shows efficacy against the microorganism at concentrations at or below its own MIC (6.25 μg/mL). Preferred embodiments include ST2-7-2, ST2-11-1, and ST2-37-1, each of which shows superior activity against resistant E. coli, with synergy indices well below 1.0 for all structural classes of antibiotics tested.

Abbreviations in Table 4 are as follows. Ab=antibiotic, Q_{A}=MIC of combination alone, Q_{B}=MIC of antibiotic alone, Qₐ=MIC of combination in conjunction with 0.5×MIC of the antibiotic, Q_{b}=Concentration of antibiotic in conjunction with test combination (0.5×MIC).

Comparative data for selected examples. Table 5 demonstrates unexpected properties of the antimicrobial compositions. For the five selected examples, none of the observed synergy among the three agents can be explained by any two-way combination of the agents. For example, the MIC of the composition ST1-73-1 is 0.39. The lowest MIC of any of the two way combinations of agents comprising the three-component compositions is 1.56. Therefore, the presence of each component is necessary to achieve the observed antimicrobial efficacy of the combination as a whole.

Example synergistic combinations are set forth below:

Combination 1

| | Component 1 | Component 2 | Component 3 |
|---|---|---|---|
| Structural | Amine or Amine | Carbohydrate or | Sequestering Agent |
| Combination | Derivative | Carbohydrate | |
| | | Derivative | |
| Example 1 | Piperine | Chitosan | KH2PO2 |
| Example 2 | Piperine | Chitosan | HEDP |
| Example 3 | Piperine | Chitosan | Salicylic Acid |

Combination 2

| | Component 1 | Component 2 | Component 3 |
|---|---|---|---|
| Structural | Amine or Amine | Amine or Amine | Sequestering Agent |
| Combination | Derivative | Derivative | |
| Example 1 | Nisin | Piperine | 8-hydroxyquinoline |
| Example 2 | Piperine | Methylene Blue | Salicylic Acid |
| Example 3 | Nisin | Piperine | Salicylic Acid |
| Example 4 | Nisin | Piperine | HEDP |
| Example 5 | Nisin | Piperine | Citric Acid |
| Example 6 | Nisin | Piperine | 2-hydroxypropyl-a- |
| | | | cyclodextrin |
| Example 7 | Nlsin | Piperine | KEDTA |

Combination 3

| | Component 1 | Component 2 | Component 3 |
|---|---|---|---|
| Structural | Terpene/Terpenoid | Amine or Amine Derivative | Sequestering Agent |
| Combination | | | |
| Example 1 | Nerolidol | N,N-bis(3- | Salicylic Acid |
| | | aminopropyl)dodecylamine | |
| Example 2 | Limonene | Piperine | 8-hydroxyquinoline |
| Example 3 | Nerolidol | Methylene Blue | HEDP |
| Example 4 | Nerolidol | Nisin | HEDP |
| Example 5 | Nerolidol | Nisin | Lactoferrin |
| Example 6 | Nerolidol | Nisin | 2-hydroxypropyl-a- |
| | | | cyclodextrin |
| Example 7 | Limonene | Piperine | HEDP |
| Example 8 | Nerolidol | CPC | 2-hydroxypropyl-a- |
| | | | cyclodextrin |
| Example 9 | Nerolidol | Na Pyrithione | 8-hydroxyquinoline |
| Example 10 | Terpene/ | Amine or Amine Derivative | Sequestering Agent |
| | Terpenoid | | |

Combination 4

| | Component 1 | Component 2 | Component 3 |
|---|---|---|---|
| Structural | Terpene/ | Terpene/Terpenoid | Sequestering Agent |
| Combination | Terpenoid | | |
| Example 1 | Nerolidol | Limonene | HEDP |
| Example 2 | Nerolidol | Linalool | Salicylic Acid |
| Example 3 | Nerolidol | Linalool | 2-hydroxypropyl-a- |
| | | | cyclodextrin |
| Example 4 | Nerolidol | Limonene | 2-hydroxypropyl-a- |
| | | | cyclodextrin |
| Example 5 | Ursolic Acid | Limonene | HEDP |
| Example 6 | Terpene/ | Terpene/Terpenoid | Sequestering Agent |
| | Terpenoid | | |

Combination 5

| | Component 1 | Component 2 | Component 3 |
|---|---|---|---|
| Structural | Terpene/ | Carbohydrate or | Sequestering Agent |
| Combination | Terpenoid | Carbohydrate | |
| | | Derivative | |
| Example 1 | Nerolidol | Octyl Glucoside | Salicylic Acid |
| Example 2 | Nerolidol | Glycerol | 2-hydroxypropyl-a- |
| | | monocaprylate | cyclodextrin |
| Example 3 | Limonene | Glycerol | Salicylic Acid |
| | | monocaprylate | |
| Example 4 | Limonene | Glycerol | HEDP |
| | | monocaprylate | |

Having now described embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention.

TABLE 1
Table 1: Proposed Embodiments

| Component 1 | Class | Component 2 | Class | Component 3 | Class | Component 4 | Class | Component 5 | Class |
|---|---|---|---|---|---|---|---|---|---|
| Nerolidol (1250 ppm) | T | Octyl glucoside (2500 ppm) | C/CD | Salicylic acid | Se | — | | — | |
| Piperine | A/AD | Chitosan | C/CD | KH2PO2 | Se | — | | — | |
| Nerolidol | T | Allyl isothiocyanate | OI | HEDP | Se | — | | — | |
| Allyl isothiocyanate | OI | N,N-bis(3- | A/AD | Salicylic acid | Se | — | | — | |
| | | aminopropyl)dodecylamine | | | | | | | |
| Nerolidol | T | N,N-bis(3- | A/AD | Salicylic acid | Se | — | | — | |
| | | aminopropyl)dodecylamine | | | | | | | |
| Nisin | A/AD | Allyl isothiocyanate | OI | HEDP | Se | — | | — | |
| Nisin | A/AD | Piperine | A/AD | 8-hydroxyquinoline | Se | — | | — | |
| Nerolidol | T | Methylene blue | A/AD | HEDP | Se | — | | — | |
| Allyl isothiocyanate | OI | Na pyrithione | A/AD | 2-hydroxypropyl-a-cyclodextrin | Se | — | | — | |
| Limonene | T | Na pyrithione | A/AD | Salicylic acid | Se | — | | — | |
| Piperine | A/AD | Allyl isothiocyanate | OI | Salicylic acid | Se | — | | — | |
| Piperine | A/AD | Limonene | T | 8-hydroxyquinoline | Se | — | | — | |
| N,N-bis(3- | A/AD | Naphthalene sulphonic acid | Su | KH2PO2 | Se | — | | — | |
| aminopropyl)dodecylamine | | | | | | | | | |
| Nerolidol | T | Usnic Acid | DD | 2-hydroxypropyl-a- | Se | — | | — | |
| | | | | cyclodextrin | | | | | |
| Allyl isothiocyanate | OI | Methylene blue | A/AD | KH2PO2 | Se | — | | — | |
| Glycerol monocaprylate | C/CD | Allyl isothiocyanate | OI | Salicylic acid | Se | — | | — | |
| Usnic Acid (0.20%) | DD | Na pyrithione | A/AD | Salicylic acid | Se | — | | — | |
| Lauryl Dimethylamine | A/AD | CPC | A/AD | K2EDTA | Se | — | | — | |
| Oxide | | | | | | | | | |
| CPC | A/AD | Usnic Acid | DD | HEDP | Se | — | | — | |
| Na pyrithione | A/AD | Naphthalene sulphonic acid | Su | Tannic Acid | P | — | | — | |
| Piperine | A/AD | Na pyrithione | A/AD | 2-hydroxypropyl-a- | Se | — | | — | |
| | | | | cyclodextrin | | | | | |
| Nerolidol | T | CPC | A/AD | Salicylic acid | Se | — | | — | |
| Allyl isothiocyanate | OI | Usnic Acid 0.2% (2000 ppm) | DD | Tannic acid | P | — | | — | |
| Phospholipid CDM | FA | Limonene | T | Salicylic acid | Se | — | | — | |
| N,N-bis(3-aminopropyl)dodecylamine | A/AD | Na pyrithione | A/AD | KH2PO2 | Se | — | | — | |
| Nisin | A/AD | Methylene blue | A/AD | K2EDTA | Se | — | | — | |
| Lauryl Dimethylamine | A/AD | Allyl isothiocyanate | OI | 8-hydroxyquinoline | Se | — | | — | |
| Oxide | | | | | | | | | |
| Piperine | A/AD | N,N-bis(3- | A/AD | 8-hydroxyquinoline | Se | — | | — | |
| | | aminopropyl)dodecylamine | | | | | | | |
| Naphthalene sulphonic acid | Su | H2O2 | Pe | HEDP | Se | — | | — | |
| CPC | A/AD | Chitosan (2500 ppm) | C/CD | 8-hydroxyquinoline (1250 ppm) | Se | — | | — | |
| Octyl glucoside | C/CD | Na pyrithione | A/AD | 8-hydroxyquinoline | Se | — | | — | |
| Phospholipid CDM | FA | Methylene blue | A/AD | K2EDTA | Se | — | | — | |
| Allyl isothiocyanate | OI | Naphthalene sulphonic acid | Su | 2-hydroxypropyl-a- | Se | — | | — | |
| | | | | cyclodextrin | | | | | |
| Nisin (1250 ppm) | A/AD | Allyl isothiocyanate (2500 ppm) | OI | Tannic acid | P | — | | — | |
| CPC | A/AD | Na pyrithione | A/AD | Tannic acid | P | — | | — | |
| Methylene blue | A/AD | Usnic Acid | DD | K2EDTA | Se | — | | — | |
| Limonene | T | Naphthalene sulphonic acid | Su | 2-hydroxypropyl-a- | Se | — | | — | |
| | | | | cyclodextrin | | | | | |
| Piperine | A/AD | Naphthalene sulphonic acid | Su | Salicylic acid | Se | — | | — | |
| Allyl isothiocyanate (2500 ppm) | OI | Limonene (2500 ppm) | T | Salicylic acid | Se | — | | — | |
| Usnic Acid 0.2% (2000 ppm) | DD | H2O2 | Pe | K2EDTA | Se | — | | — | |
| Nerolidol (1250 ppm) | T | Piperine (1250 ppm) | A/AD | Octyl glucoside (2500 ppm) | C/CD | — | | — | |
| Nerolidol | T | Na pyrithione (1250 ppm) | A/AD | HEDP | Se | — | | — | |
| Methylene blue | A/AD | Naphthalene sulphonic acid | Su | K2EDTA | Se | — | | — | |
| Glycerol monocaprylate | C/CD | Methylene blue | A/AD | Tannic acid | P | — | | — | |
| Phospholipid CDM | FA | Naphthalene sulphonic acid | Su | K2EDTA | Se | — | | — | |
| Phospholipid CDM (1250 ppm) | FA | Nerolidol (0.125) | T | Tannic acid | P | — | | — | |
| Piperine | A/AD | Methylene blue | A/AD | Salicylic acid | Se | — | | — | |
| Piperine | A/AD | Usnic Acid | DD | Salicylic acid | Se | — | | — | |
| Lauryl Dimethylamine | A/AD | Chitosan (2500 ppm) | C/CD | Salicylic acid | Se | — | | — | |
| Oxide (1250 ppm) | | | | | | | | | |
| Piperine | A/AD | Carvacrol | P | 2-hydroxypropyl-a- | Se | — | | — | |
| | | | | cyclodextrin | | | | | |
| Usnic Acid 0.2% (2000 ppm) | DD | N,N-bis(3- | A/AD | Na pyrophosphate | Se | — | | — | |
| | | aminopropyl)dodecylamine | | | | | | | |
| Na lignosulphonate | Su | Na pyrithione | A/AD | HEDP | Se | — | | — | |
| L-carnitine | Q | Na pyrithione | A/AD | Na pyrophosphate | Se | — | | — | |
| Nisin | A/AD | Carvacrol | P | 2-hydroxypropyl-a- | Se | — | | — | |
| | | | | cyclodextrin | | | | | |
| Allyl isothiocyanate | OI | Totarol | T | 8-hydroxyquinoline | Se | — | | — | |
| Activin | P | Methylene blue | A/AD | 8-hydroxyquinoline | Se | — | | — | |
| L-carnitine (2500 ppm) | A/AD | H2O2 | Pe | HEDP | Se | — | | — | |
| Nisin (1250 ppm) | A/AD | L-carnitine (2500 ppm) | Q | N,N-bis(3-aminopropyl)dodecylamine | A/AD | — | | — | |
| | | | | (2500 ppm) | | | | | |
| Chitosan | C/CD | Limonene | T | Salicyclic acid | Se | — | | — | |
| Activin | P | L-carnitine | Q | 8-hydroxyquinoline | Se | — | | — | |
| Piperine | A/AD | L-carnitine | Q | KH2PO2 | Se | — | | — | |
| Carvacrol | P | Methylene blue | A/AD | 2-hydroxypropyl-a- | Se | — | | — | |
| | | | | cyclodextrin | | | | | |
| Nerolidol (1250 ppm) | T | Carvacrol (2500 ppm) | P | Salicylic acid | Se | — | | — | |
| Totarol 0.25% (2500 ppm) | T | Naphthalene sulphonic acid 0.25% | Su | HEDP | Se | — | | — | |
| | | (2500 ppm) | | | | | | | |
| Octyl gallate | P | Naphthalene sulphonic acid | Su | Na pyrophosphate | Se | — | | — | |
| Piperine (1250 ppm) | A/AD | Activin (2500 ppm) | P | 8-hydroxyquinoline (1250 ppm) | Se | — | | — | |
| Piperine (1250 ppm) | A/AD | Octyl gallate (2500 ppm) | P | Limonene (2500 ppm) | T | — | | — | |
| Na lignosulphonate | Su | L-carnitine | Q | K2EDTA | Se | — | | — | |
| Phospholipid CDM (1250 ppm) | FA | Octyl gallate (2500 ppm) | P | 2-hydroxypropyl-a- | Se | — | | — | |
| | | | | cyclodextrin | | | | | |
| Lauryl Dimethylamine | A/AD | Nisin (1250 ppm) | A/AD | Na pyrophosphate | Se | — | | — | |
| Oxide (1250 ppm) | | | | | | | | | |
| Methylene blue | A/AD | N,N-bis(3- | A/AD | Na pyrophosphate | Se | — | | — | |
| | | aminopropyl)dodecylamine | | | | | | | |
| Activin | P | Chitosan | C/CD | H3PO2 | Se | — | | — | |
| Na lignosulphonate | Su | Naphthalene sulphonic acid (2500 ppm) | Su | HEDP | Se | — | | — | |
| Na lignosulphonate | Su | Naphthalene sulphonic acid | Su | Na pyrophosphate | Se | — | | — | |
| Na lignosulphonate | Su | Octyl glucoside | C/CD | Tannic acid | P | — | | — | |
| Nerolidol | T | HEDP | Se | Limonene | T | — | | — | |
| Nerolidol | T | HEDP | Se | Thymol | P | — | | — | |
| Thymol | P | Citric Acid | Se | Limonene | T | — | | — | |
| Nisin | A/AD | Piperine | A/AD | Salicylic Acid | Se | — | | — | |
| Nisin | A/AD | Piperine | A/AD | HEDP | Se | — | | — | |
| Nisin | A/AD | Piperine | A/AD | Citric Acid | Se | — | | — | |
| Nisin | A/AD | Piperine | A/AD | 2-hydroxypropyl-a- | Se | — | | — | |
| | | | | cyclodextrin | | | | | |
| Nisin | A/AD | Piperine | A/AD | KEDTA | Se | — | | — | |
| Chitosan | C/CD | Citric Acid | Se | Salicylic Acid | Se | — | | — | |
| Chitosan | C/CD | Citric Acid | Se | HEDP | Se | — | | — | |
| Chitosan | C/CD | Citric Acid | Se | 2-hydroxypropyl-a- | Se | — | | — | |
| | | | | cyclodextrin | | | | | |
| Nerolidol | T | 2-hydroxypropyl-a-cyclodextrin | Se | Thymol | P | — | | — | |
| Nerolidol | T | 2-hydroxypropyl-a-cyclodextrin | Se | Glycerol monocaprylate | C/CD | — | | — | |
| Nerolidol | T | 2-hydroxypropyl-a-cyclodextrin | Se | Linalool | T | — | | — | |
| Nerolidol | T | 2-hydroxypropyl-a-cyclodextrin | Se | Limonene | T | — | | — | |
| Nerolidol | T | Linalool | T | Salicylic acid | Se | — | | — | |
| Piperine | A/AD | Thymol | P | Salicylic acid | Se | — | | — | |
| Piperine | A/AD | Thymol | P | HEDP | Se | — | | — | |
| Piperine | A/AD | Chitosan | C/CD | HEDP | Se | — | | — | |
| Nisin | A/AD | HEDP | Se | Nerolidol | T | — | | — | |
| Nisin | A/AD | Lactoferrin | Se | Nerolidol | T | — | | — | |
| Nisin | A/AD | 2-hydroxypropyl-a-cyclodextrin | Se | Nerolidol | T | — | | — | |
| Piperine | A/AD | Limonene | T | HEDP | Se | — | | — | |
| Glycerol monocaprylate | C/CD | Salicylic acid | Se | Limonene | T | — | | — | |
| Glycerol monocaprylate | C/CD | HEDP | Se | Limonene | T | — | | — | |
| Glycerol monocaprylate | C/CD | 2-hydroxypropyl-a-cyclodextrin | Se | Limonene | T | — | | — | |
| Naphthalene sulphonic acid | Su | HEDP | Se | Limonene | T | — | | — | |
| Naphthalene sulphonic acid | Su | HEDP | Se | Glycerol monocaprylate | C/CD | — | | — | |
| Ursolic Acid | T | HEDP | Se | Limonene | T | — | | — | |
| N,N-bis(3- | A/AD | Naphthalene Sulfonic Acid | Su | HEDP | Se | — | | — | |
| aminopropyl)dodecylamine | | | | | | | | | |
| CPC | A/AD | Nerolidol | T | 2-hydroxypropyl-a- | Se | — | | — | |
| | | | | cyclodextrin | | | | | |
| Nerolidol | T | Na pyrithione | A/AD | 8-hydroxyquinoline | Se | — | | — | |
| Piperine | A/AD | Chitosan | C/CD | Salicylic acid | Se | — | | — | |
| Catechins | P | Nisin | A/AD | HEDP | Se | — | | — | |
| Catechins | P | Chistosan | C/CD | 2-hydroxypropyl-a- | Se | — | | — | |
| | | | | cyclodextrin | | | | | |
| Catechins | P | Nerolidol | T | Salicylic acid | Se | — | | — | |
| Propolis | Prop | Piperine | A/AD | Salicylic acid | Se | — | | — | |
| Propolis | Prop | Phospholipid CDM | FA | 2-hydroxypropyl-a- | Se | — | | — | |
| | | | | cyclodextrin | | | | | |
| Natamycin | MP | Nerolidol | T | HEDP | Se | — | | — | |
| Natamycin | MP | Limonene | T | Salicylic acid | Se | — | | — | |
| Sodium carbonate | Pe | Alkyl dimethyl benzyl ammonium | A/AD | Citric Acid | Se | — | | — | |
| peroxyhydrate | | chloride | | | | | | | |
| Hydrogen Peroxide | Pe | Alkyl dimethyl benzyl ammonium | Q | Na4EDTA | Se | Nerolidol | T | — | |
| | | chloride | | | | | | | |
| Hydrogen Peroxide | Pe | Alkyl dimethyl benzyl ammonium | Q | Na4EDTA | Se | Piperine | A/AD | — | |
| | | chloride | | | | | | | |
| Hydrogen Peroxide | Pe | Alkyl dimethyl benzyl ammonium | Q | Na4EDTA | Se | Nerolidol | T | Piperine | A/AD |
| | | chloride | | | | | | | |
| Hydrogen Peroxide | Pe | Alkyl dimethyl benzyl ammonium | Q | Na4EDTA | Se | Sodium | Su | — | |
| | | chloride | | | | lignosulfonate | | | |
| Hydrogen Peroxide | Pe | Alkyl dimethyl benzyl ammonium | Q | Na4EDTA | Se | Carvacrol | P | — | |
| | | chloride | | | | | | | |
| Hydrogen Peroxide | Pe | Alkyl dimethyl benzyl ammonium | Q | Na4EDTA | Se | Chitosan | C/CD | — | |
| | | chloride | | | | | | | |
| Hydrogen Peroxide | Pe | Alkyl dimethyl benzyl ammonium | Q | Na4EDTA | Se | L-carnitine | Q | — | |
| | | chloride | | | | | | | |
| Hydrogen Peroxide | Pe | Alkyl dimethyl benzyl ammonium | Q | Na4EDTA | Se | Limonene | T | — | |
| | | chloride | | | | | | | |
| Hydrogen Peroxide | Pe | Alkyl dimethyl benzyl ammonium | Q | Na4EDTA | Se | Naphthalene | Su | — | |
| | | chloride | | | | sulfonic acid | | | |
| Hydrogen Peroxide | Pe | Alkyl dimethyl benzyl ammonium | Q | Na4EDTA | Se | Thymol | P | — | |
| | | chloride | | | | | | | |
| Hydrogen Peroxide | Pe | Alkyl dimethyl benzyl ammonium | Q | Na4EDTA | Se | Linalool | T | — | |
| | | chloride | | | | | | | |
| Hydrogen Peroxide | Pe | Alkyl dimethyl benzyl ammonium | Q | Na4EDTA | Se | Glycerol | C/CD | — | |
| | | chloride | | | | monocaprylate | | | |
| Hydrogen Peroxide | Pe | Alkyl dimethyl benzyl ammonium | Q | Nerolidol | T | HEDP | Se | — | |
| | | chloride | | | | | | | |
| Hydrogen Peroxide | Pe | Alkyl dimethyl benzyl ammonium | Q | Nerolidol | T | K2EDTA | Se | — | |
| | | chloride | | | | | | | |
| Hydrogen Peroxide | Pe | Alkyl dimethyl benzyl ammonium | Q | Nerolidol | T | 2-hydroxypropyl-a- | Se | — | |
| | | chloride | | | | cyclodextrin | | | |
| Hydrogen Peroxide | Pe | Alkyl dimethyl benzyl ammonium | Q | Nerolidol | T | Citric Acid | Se | — | |
| | | chloride | | | | | | | |
| Hydrogen Peroxide | Pe | Alkyl dimethyl benzyl ammonium | Q | Piperine | A/AD | HEDP | Se | — | |
| | | chloride | | | | | | | |
| Hydrogen Peroxide | Pe | Alkyl dimethyl benzyl ammonium | Q | Piperine | A/AD | K2EDTA | Se | — | |
| | | chloride | | | | | | | |
| Hydrogen Peroxide | Pe | Alkyl dimethyl benzyl ammonium | Q | Piperine | A/AD | 2-hydroxypropyl-a- | Se | — | |
| | | chloride | | | | cyclodextrin | | | |
| Hydrogen Peroxide | Pe | Alkyl dimethyl benzyl ammonium | Q | Piperine | A/AD | Citric Acid | Se | — | |
| | | chloride | | | | | | | |
| Hydrogen Peroxide | Pe | Alkyl dimethyl benzyl ammonium | Q | Carvacrol | P | HEDP | Se | — | |
| | | chloride | | | | | | | |
| Hydrogen Peroxide | Pe | Alkyl dimethyl benzyl ammonium | Q | Carvacrol | P | K2EDTA | Se | — | |
| | | chloride | | | | | | | |
| Hydrogen Peroxide | Pe | Alkyl dimethyl benzyl ammonium | Q | Carvacrol | P | 2-hydroxypropyl-a- | Se | — | |
| | | chloride | | | | cyclodextrin | | | |
| Hydrogen Peroxide | Pe | Alkyl dimethyl benzyl ammonium | Q | Citric Acid | Se | — | | — | |
| | | chloride | | | | | | | |
| Hydrogen Peroxide | Pe | Alkyl dimethyl benzyl ammonium | Q | Glycerol monocaprylate | C/CD | K2EDTA | Se | — | |
| | | chloride | | | | | | | |
| Hydrogen Peroxide | Pe | Alkyl dimethyl benzyl ammonium | Q | Glycerol monocaprylate | C/CD | HEDP | Se | — | |
| | | chloride | | | | | | | |
| Hydrogen Peroxide | Pe | Alkyl dimethyl benzyl ammonium | Q | Glycerol monocaprylate | C/CD | Citric Acid | Se | — | |
| | | chloride | | | | | | | |
| Hydrogen Peroxide | Pe | Alkyl dimethyl benzyl ammonium | Q | Limonene | T | HEDP | Se | — | |
| | | chloride | | | | | | | |
| Hydrogen Peroxide | Pe | Alkyl dimethyl benzyl ammonium | Q | Limonene | T | K2EDTA | Se | — | |
| | | chloride | | | | | | | |
| Hydrogen Peroxide | Pe | Alkyl dimethyl benzyl ammonium | Q | Limonene | T | 2-hydroxypropyl-a- | Se | — | |
| | | chloride | | | | cyclodextrin | | | |
| Hydrogen Peroxide | Pe | Alkyl dimethyl benzyl ammonium | Q | Thymol | P | Citric Acid | Se | — | |
| | | chloride | | | | | | | |
| Hydrogen Peroxide | Pe | Alkyl dimethyl benzyl ammonium | Q | 2-hydroxypropyl-a- | Se | Thymol | P | — | |
| | | chloride | | cyclodextrin | | | | | |
| Hydrogen Peroxide | Pe | Alkyl dimethyl benzyl ammonium | Q | HEDP | Se | Thymol | P | — | |
| | | chloride | | | | | | | |
| Hydrogen Peroxide | Pe | Na4EDTA | Se | Nisin | A/AD | — | | — | |
| Hydrogen Peroxide | Pe | Na4EDTA | Se | Nerolidol | T | — | | — | |
| Hydrogen Peroxide | Pe | Na4EDTA | Se | Piperine | A/AD | — | | — | |
| Hydrogen Peroxide | Pe | Na4EDTA | Se | Sodium lignosulfonate | Su | — | | — | |
| Hydrogen Peroxide | Pe | Na4EDTA | Se | Nerolidol | T | CPC | A/AD | — | |
| Hydrogen Peroxide | Pe | Na4EDTA | Se | Piperine | A/AD | CPC | A/AD | — | |
| Hydrogen Peroxide | Pe | Na4EDTA | Se | CPC | A/AD | Carvacrol | P | — | |
| Hydrogen Peroxide | Pe | Na4EDTA | Se | CPC | A/AD | Totarol | T | Limonene | T |
| Hydrogen Peroxide | Pe | Na4EDTA | Se | CPC | A/AD | — | | — | |
| Hydrogen Peroxide | Pe | Na4EDTA | Se | CPC | A/AD | Thymol | P | — | |
| Hydrogen Peroxide | Pe | Na4EDTA | Se | CPC | A/AD | Linalool | T | — | |
| Hydrogen Peroxide | Pe | Na4EDTA | Se | Natamycin | MP | — | | — | |
| Hydrogen Peroxide | Pe | Na4EDTA | Se | CPC | A/AD | Limonene | T | — | |
| Hydrogen Peroxide | Pe | Na4EDTA | Se | Catechin | P | — | | — | |
| Hydrogen Peroxide | Pe | Alkyl dimethyl benzyl ammonium | Q | Catechin | P | — | | — | |
| | | chloride | | | | | | | |
| Hydrogen Peroxide | Pe | Na4EDTA | Se | CPC | A/AD | Glycerol | C/CD | — | |
| | | | | | | monocaprylate | | | |
| Hydrogen Peroxide | Pe | Na4EDTA | Se | Chitosan | C/CD | — | | — | |
| Hydrogen Peroxide | Pe | Na4EDTA | Se | Octyl glucoside | C/CD | — | | — | |
| Hydrogen Peroxide | Pe | Na4EDTA | Se | Octyl gallate | P | — | | — | |
| Hydrogen Peroxide | Pe | Alkyl dimethyl benzyl ammonium | Q | CPC | A/AD | Lactoferrin | Se | — | |
| | | chloride | | | | | | | |
| Hydrogen Peroxide | Pe | Alkyl dimethyl benzyl ammonium | Q | Na4EDTA | Se | Lactoferrin | Se | — | |
| | | chloride | | | | | | | |
| Hydrogen Peroxide | Pe | Alkyl dimethyl benzyl ammonium | Q | Lactoferrin | Se | Thymol | P | — | |
| | | chloride | | | | | | | |
| Hydrogen Peroxide | Pe | Alkyl dimethyl benzyl ammonium | Q | Nerolidol | T | Lactoferrin | Se | — | |
| | | chloride | | | | | | | |
| Hydrogen Peroxide | Pe | Alkyl dimethyl benzyl ammonium | Q | Limonene | T | Lactoferrin | Se | — | |
| | | chloride | | | | | | | |
| Hydrogen Peroxide | Pe | Na4EDTA | Se | CPC | A/AD | Citric Acid | Se | — | |
| Hydrogen Peroxide | Pe | Na4EDTA | Se | CPC | A/AD | Citric Acid | Se | Linalool | T |
| Hydrogen Peroxide | Pe | Na4EDTA | Se | Citric Acid | Se | Natamycin | MP | — | |
| Hydrogen Peroxide | Pe | Alkyl dimethyl benzyl ammonium | Q | K2EDTA | Se | | | | |
| | | chloride | | | | | | | |
| Hydrogen Peroxide | Pe | Alkyl dimethyl benzyl ammonium | Q | Citric Acid | Se | | | | |
| | | chloride | | | | | | | |
| Hydrogen Peroxide | Pe | Alkyl dimethyl benzyl ammonium | Q | 2-hydroxypropyl-a- | Se | | | | |
| | | chloride | | cyclodextrin | | | | | |

T = Terpene/Terpenoid
A/AD = Amine/Amine Derivative
Se = Sequestrant
C/CD = Carbohydrate/Carbohydrate Derivative
Prop = Propolis
DD = Dibenzofuran Derivative
Su = Sulfonate
OI = Organo isothiocyanate
P = Phenol
FA = Fatty acid
Pe = Peroxide/Peroxide Donor
Q = Quaternary Ammonium Compound
MP = Macrolide Polyene

TABLE 2
Table 2: Synergistic combinations - MIC data against MRSA

| | MIC of | | 1-MIC- | | 2-MIC- |
|---|---|---|---|---|---|
| Sample | comb.- | Component 1 | MRSA | Component 2 | MRSA |
| ST1-72-1 | 1.56 | Lauryl Dimethylamine | 12.5 | CPC | 3.15 |
| | | Oxide | | | |
| ST1-73-1 | 0.39 | Nisin | 1001 | Piperine | 1001 |
| ST1-73-3 | 50 | Nisin | 1001 | Allyl isothiocyanate | 1001 |
| ST1-76-3 | 12.5 | Nerolidol | 500 | Allyl isothiocyanate | 1001 |
| ST1-76-4 | 6.25 | Nerolidol | 500 | Methylene blue | 25 |
| ST1-78-1 | 3.15 | Nerolidol | 500 | Usnic Acid | 6.25 |
| ST1-78-3 | 100 | Piperine | 1001 | Allyl isothiocyanate | 1001 |
| ST1-95-2 | 0.79 | Piperine | 1001 | Limonene | 1001 |
| ST2-11-1 | 12.5 | Piperine | 1001 | Chitosan | 1001 |
| ST2-14-2 | 12.5 | Allyl isothiocyanate | 1001 | Methylene blue | 25 |
| ST2-3-2 | 1.56 | CPC | 3.15 | Usnic Acid | 6.25 |
| ST2-34-1 | 0.79 | Allyl isothiocyanate | 1001 | N,N-bis(3- | 12.5 |
| | | | | aminopropyl)dodecylamine | |
| ST2-34-2 | 0.39 | Usnic Acid (0.20%) | 6.25 | Na pyrithione | 0.79 |
| ST2-37-1 | 1.56 | Nerolidol | 500 | N,N-bis(3- | 12.5 |
| | | | | aminopropyl)dodecylamine | |
| ST2-39-1 | 6.25 | N,N-bis(3- | 12.5 | Naphthalene sulphonic acid | 1001 |
| | | aminopropyl)dodecylamine | | | |
| ST2-55-1 | 1.56 | Nerolidol (1250 ppm) | 500 | Octyl glucoside (2500 ppm) | 1001 |
| ST2-6-2 | 25 | Glycerol monocaprylate | 50 | Allyl isothiocyanate | 1001 |
| ST2-7-2 | 0.39 | Allyl isothiocyanate | 1001 | Na pyrithione | 0.79 |
| ST2-8-2 | 0.39 | Limonene | 1001 | Na pyrithione | 0.79 |

| | | 3-MIC- | | Other- | SI- | SI- |
|---|---|---|---|---|---|---|
| Sample | Component 3 | MRSA | Other | MIC- | MRSA | MRSA- |
| ST1-72-1 | K2EDTA | 500 | Phenoxyethanol | 6080 | 0.623 | 0.623 |
| ST1-73-1 | 8-hydroxyquinoline | 1.56 | Phenoxyethanol | 6080 | 0.251 | 0.251 |
| ST1-73-3 | HEDP | 1001 | Phenoxyethanol | 6080 | 0.150 | 0.158 |
| ST1-76-3 | HEDP | 1001 | Phenoxyethanol | 6080 | 0.050 | 0.052 |
| ST1-76-4 | HEDP | 1001 | Phenoxyethanol | 6080 | 0.269 | 0.270 |
| ST1-78-1 | 2-hydroxypropyl-α- | 1001 | Phenoxyethanol | 6080 | 0.513 | 0.514 |
| | cyclodextrin | | | | | |
| ST1-78-3 | Salicylic acid | 1001 | THFA | 500 | 0.300 | 0.500 |
| ST1-95-2 | 8-hydroxyquinoline | 1.56 | Phenoxyethanol | 6080 | 0.508 | 0.508 |
| ST2-11-1 | KH2PO2 | 1001 | pluronic F127 | 1001 | 0.037 | 0.050 |
| ST2-14-2 | KH2PO2 | 1001 | pluronic F127 | 1001 | 0.525 | 0.537 |
| ST2-3-2 | HEDP | 1001 | Phenoxyethanol | 6080 | 0.746 | 0.747 |
| ST2-34-1 | Salicylic acid | 1001 | PEG 400 | 1001 | 0.065 | 0.066 |
| ST2-34-2 | Salicylic acid | 1001 | PEG 400 | 1001 | 0.556 | 0.557 |
| ST2-37-1 | Salicylic acid | 1001 | PEG 400 | 1001 | 0.129 | 0.131 |
| ST2-39-1 | KH2PO2 | 1001 | H₂O | 0 | 0.512 | 0.512 |
| ST2-55-1 | Salicylic acid | 1001 | PEG 400 | 1001 | 0.006 | 0.008 |
| ST2-6-2 | Salicylic acid | 1001 | Phenoxyethanol | 6080 | 0.550 | 0.554 |
| ST2-7-2 | 2-hydroxypropyl-α- | 1001 | Phenoxyethanol | 6080 | 0.494 | 0.495 |
| | cyclodextrin | | | | | |
| ST2-8-2 | Salicylic acid | 1001 | Phenoxyethanol | 6080 | 0.494 | 0.495 |

TABLE 3
Table 3: Synergistic combinations - MIC data against E. coli

| | MIC of | | | | | | | | Other- | | SI- |
|---|---|---|---|---|---|---|---|---|---|---|---|
| | comb.- | | 1- | | 2- | | 3- | | MIC- | SI- | ecoli- |
| Sample | ecoli | Component 1 | MIC- | Component 2 | MIC- | Component 3 | MIC- | Other | ecoli | ecoli | other |
| ST1-72-2 | 12 | Lauryl | 1001 | Allyl isothiocyanate | 1000 | 8- | 50 | H₂O | 0 | 0.264 | 0.264 |
| | | Dimethylamine | | | | hydroxyquinoline | | | | | |
| | | Oxide | | | | | | | | | |
| ST1-73-1 | 6 | Nisin | 1001 | Piperine | 1001 | 8- | 50 | Phenoxyethanol | 3200 | 0.132 | 0.134 |
| ST1-73-3 | 25 | Nisin | 1001 | Allyl isothiocyanate | 1000 | HEDP | 1001 | Phenoxyethanol | 3200 | 0.075 | 0.083 |
| ST1-76-1 | 12 | Phospholipid | 25 | Limonene | 1001 | Salicylic acid | 1001 | Phenoxyethanol | 3200 | 0.504 | 0.508 |
| | | CDM | | | | | | | | | |
| ST1-76-2 | 2 | Nerolidol | 1001 | CPC | 3 | Salicylic acid | 1001 | Phenoxyethanol | 3200 | 0.671 | 0.671 |
| ST1-76-3 | 12 | Nerolidol | 1001 | Allyl isothiocyanate | 1000 | HEDP | 1001 | Phenoxyethanol | 3200 | 0.036 | 0.040 |
| ST1-76-4 | 12 | Nerolidol | 1001 | Methylene blue | 1001 | HEDP | 1001 | Phenoxyethanol | 3200 | 0.036 | 0.040 |
| ST1-78-1 | 6 | Nerolidol | 1001 | Usnic Acid | 1001 | 2-hydroxypropyl- | 1001 | Phenoxyethanol | 3200 | 0.018 | 0.020 |
| | | | | | | α-cyclodextrin | | | | | |
| ST1-92-1 | 25 | Piperine | 1001 | Methylene blue | 1001 | Salicylic acid | 1001 | Phenoxyethanol | 3200 | 0.075 | 0.083 |
| ST1-93-1 | 6.25 | Piperine | 1001 | N,N-bis(3- | 25 | 8- | 50 | Phenoxyethanol | 3200 | 0.381 | 0.383 |
| | | | | aminopropyl)dodecylamine | | hydroxyquinoline | | | | | |
| ST1-95-2 | 25 | Piperine | 1001 | Limonene | 1001 | 8- | 50 | Phenoxyethanol | 3200 | 0.550 | 0.558 |
| ST2-11-1 | 50 | Piperine | 1001 | Chitosan | 1001 | KH2PO2 | 1001 | pluronic F127 | 1001 | 0.150 | 0.200 |
| ST2-15-1 | 100 | Methylene | 1001 | Usnic Acid | 1001 | K2EDTA | 1000 | pluronic F127 | 1001 | 0.300 | 0.400 |
| | | blue | | | | | | | | | |
| ST2-34-1 | 1.56 | Allyl | 1000 | N,N-bis(3- | 25 | Salicylic acid | 1001 | PEG 400 | 1001 | 0.066 | 0.067 |
| | | isothiocyanate | | aminopropyl)dodecylamine | | | | | | | |
| ST2-34-2 | 1.56 | Usnic Acid | 1001 | Na pyrithione | 6.25 | Salicylic acid | 1001 | PEG 400 | 1001 | 0.253 | 0.254 |
| | | (0.20%) | | | | | | | | | |
| ST2-37-1 | 6.25 | Nerolidol | 1001 | N,N-bis(3- | 25 | Salicylic acid | 1001 | PEG 400 | 1001 | 0.262 | 0.269 |
| | | | | aminopropyl)dodecylamine | | | | | | | |
| ST2-39-1 | 12.5 | N,N-bis(3- | 25 | Naphthalene sulphonic acid | 1001 | KH2PO2 | 1001 | H2O | 0 | 0.525 | 0.525 |
| | | amino- | | | | | | | | | |
| | | propyl)do- | | | | | | | | | |
| | | decylamine | | | | | | | | | |
| ST2-6-2 | 12.5 | Glycerol | 1001 | Allyl isothiocyanate | 1000 | Salicylic acid | 1001 | Phenoxyethanol | 3200 | 0.037 | 0.041 |
| | | monocaprylate | | | | | | | | | |
| ST2-7-2 | 1.56 | Allyl | 1000 | Na pyrithione | 6.25 | 2-hydroxypropyl- | 1001 | Phenoxyethanol | 3200 | 0.253 | 0.253 |
| | | isothiocyanate | | | | a-cyclodextrin | | | | | |
| ST2-8-1 | 3.15 | N,N-bis(3- | 25 | Na pyrithione | 6.25 | KH2PO2 | 1001 | H₂O | 0 | 0.633 | 0.633 |
| | | amino- | | | | | | | | | |
| | | propyl)do- | | | | | | | | | |
| | | decylamine | | | | | | | | | |
| ST2-8-2 | 3.15 | Limonene | 1000 | Na pyrithione | 6.25 | Salicylic acid | 1001 | Phenoxyethanol | 3200 | 0.510 | 0.511 |

TABLE 4
Table 4: Additional synergy data

| | | | | | | MIC of | | [A] in comb. | | |
|---|---|---|---|---|---|---|---|---|---|---|
| | | | | | | comb. | MIC Ab | which | [B] in | |
| | Minimum concentration (μg/ml) of the combination that, | | | | | alone | alone | yielded | comb. | |
| Test | coupled with 0.5X MIC of antibiotic (Ab), yielded no growth | | | | | (A) | (B) | no growth | (0.5X MIC) | Qa/QA |
| Compound | Gentamicin | Tetracycline | Doxycycline | Ciprofloxacin | Ab | QA | QB | Qa | Qb | SI |
| ST1-73-1 | 0.1 | 6.25 | 6.25 | 25 | gent | 25 | 100 | 0.1 | 50 | 0.00 |
| | | | | | tetra | 25 | 400 | 6.25 | 200 | 0.25 |
| | | | | | doxy | 25 | 50 | 6.25 | 25 | 0.25 |
| | | | | | cipro | 25 | 200 | 25 | 100 | 1.00 |
| ST1-73-3 | 0.1 | 12.5 | 6.25 | 50 | gent | 50 | 100 | 0.1 | 50 | 0.00 |
| | | | | | tetra | 50 | 400 | 12.5 | 200 | 0.25 |
| | | | | | doxy | 50 | 50 | 6.25 | 25 | 0.13 |
| | | | | | cipro | 50 | 200 | 50 | 100 | 1.00 |
| ST1-76-3 | 0.1 | 12.5 | 6.25 | 50 | gent | 25 | 100 | 0.1 | 50 | 0.00 |
| | | | | | tetra | 25 | 400 | 12.5 | 200 | 0.50 |
| | | | | | doxy | 25 | 50 | 6.25 | 25 | 0.25 |
| | | | | | cipro | 25 | 200 | 50 | 100 | 2.00 |
| ST1-76-4 | 0.1 | 6.25 | 6.25 | 25 | gent | 25 | 100 | 0.1 | 50 | 0.00 |
| | | | | | tetra | 25 | 400 | 6.25 | 200 | 0.25 |
| | | | | | doxy | 25 | 50 | 6.25 | 25 | 0.25 |
| | | | | | cipro | 25 | 200 | 25 | 100 | 1.00 |
| ST1-78-1 | 0.1 | 12.5 | 12.5 | 50 | gent | 50 | 100 | 0.1 | 50 | 0.00 |
| | | | | | tetra | 50 | 400 | 12.5 | 200 | 0.25 |
| | | | | | doxy | 50 | 50 | 12.5 | 25 | 0.25 |
| | | | | | cipro | 50 | 200 | 50 | 100 | 1.00 |
| ST1-92-1 | 1.56 | 6.25 | 12.5 | 25 | gent | 25 | 100 | 1.56 | 50 | 0.06 |
| | | | | | tetra | 25 | 400 | 6.25 | 200 | 0.25 |
| | | | | | doxy | 25 | 50 | 12.5 | 25 | 0.50 |
| | | | | | cipro | 25 | 200 | 25 | 100 | 1.00 |
| ST1-93-1 | 0.1 | 1.56 | 1.56 | 12.5 | gent | 12.5 | 100 | 0.1 | 50 | 0.01 |
| | | | | | tetra | 12.5 | 400 | 1.56 | 200 | 0.12 |
| | | | | | doxy | 12.5 | 50 | 1.56 | 25 | 0.12 |
| | | | | | cipro | 12.5 | 200 | 12.5 | 100 | 1.00 |
| ST1-95-2 | 0.1 | 6.25 | 3.15 | 25 | gent | 25 | 100 | 0.1 | 50 | 0.00 |
| | | | | | tetra | 25 | 400 | 6.25 | 200 | 0.25 |
| | | | | | doxy | 25 | 50 | 3.15 | 25 | 0.13 |
| | | | | | cipro | 25 | 200 | 25 | 100 | 1.00 |
| ST2-6-2 | 0.1 | 12.5 | 6.25 | 50 | gent | 25 | 100 | 0.1 | 50 | 0.00 |
| | | | | | tetra | 25 | 400 | 12.5 | 200 | 0.50 |
| | | | | | doxy | 25 | 50 | 6.25 | 25 | 0.25 |
| | | | | | cipro | 25 | 200 | 50 | 100 | 2.00 |
| ST2-7-2 | 0.1 | 3.15 | 1.56 | 3.15 | gent | 6.25 | 100 | 0.1 | 50 | 0.02 |
| | | | | | tetra | 6.25 | 400 | 3.15 | 200 | 0.50 |
| | | | | | doxy | 6.25 | 50 | 1.56 | 25 | 0.25 |
| | | | | | cipro | 6.25 | 200 | 3.15 | 100 | 0.50 |
| ST2-8-2 | 0.1 | 6.25 | 3.15 | 6.25 | gent | 6.25 | 100 | 0.1 | 50 | 0.02 |
| | | | | | tetra | 6.25 | 400 | 6.25 | 200 | 1.00 |
| | | | | | doxy | 6.25 | 50 | 3.15 | 25 | 0.50 |
| | | | | | cipro | 6.25 | 200 | 6.25 | 100 | 1.00 |
| ST2-11-1 | 0.1 | 51 | 50 | 51 | gent | 1001 | 100 | 0.1 | 50 | 0.00 |
| | | | | | tetra | 1001 | 400 | 51 | 200 | 0.05 |
| | | | | | doxy | 1001 | 50 | 50 | 25 | 0.05 |
| | | | | | cipro | 1001 | 200 | 51 | 100 | 0.05 |
| ST2-15-1 | 0.1 | 50 | 25 | 50 | gent | 500 | 100 | 0.1 | 50 | 0.00 |
| | | | | | tetra | 500 | 400 | 50 | 200 | 0.10 |
| | | | | | doxy | 500 | 50 | 25 | 25 | 0.05 |
| | | | | | cipro | 500 | 200 | 50 | 100 | 0.10 |
| ST2-34-1 | 0.1 | 3.15 | 1.56 | 3.15 | gent | 6.25 | 100 | 0.1 | 50 | 0.02 |
| | | | | | tetra | 6.25 | 400 | 3.15 | 200 | 0.50 |
| | | | | | doxy | 6.25 | 50 | 1.56 | 25 | 0.25 |
| | | | | | cipro | 6.25 | 200 | 3.15 | 100 | 0.50 |
| ST2-34-2 | 0.1 | 3.15 | 1.56 | 3.15 | gent | 6.25 | 100 | 0.1 | 50 | 0.02 |
| | | | | | tetra | 6.25 | 400 | 3.15 | 200 | 0.50 |
| | | | | | doxy | 6.25 | 50 | 1.56 | 25 | 0.25 |
| | | | | | cipro | 6.25 | 200 | 3.15 | 100 | 0.50 |
| ST2-37-1 | 0.1 | 1.56 | 0.79 | 6.25 | gent | 12.5 | 100 | 0.1 | 50 | 0.01 |
| | | | | | tetra | 12.5 | 400 | 1.56 | 200 | 0.12 |
| | | | | | doxy | 12.5 | 50 | 0.79 | 25 | 0.06 |
| | | | | | cipro | 12.5 | 200 | 6.25 | 100 | 0.50 |
| ST2-55-1 | 0.1 | 51 | 51 | 51 | gent | 1001 | 100 | 0.1 | 50 | 0.00 |
| | | | | | tetra | 1001 | 400 | 51 | 200 | 0.05 |
| | | | | | doxy | 1001 | 50 | 51 | 25 | 0.05 |
| | | | | | cipro | 1001 | 200 | 51 | 100 | 0.05 |

TABLE 5
Table 5: Comparative data for selected examples

| | | | | | 2- | | |
|---|---|---|---|---|---|---|---|
| | MIC- | | 1-MIC- | | MIC- | | 3-MIC- |
| Sample | MRSA | Comp. 1 | MRSA | Component 2 | MRSA | Component 3 | MRSA |
| ST1-73-1 | 0.39 | Nisin | 1001 | Piperine | 1001 | 8-hydroxy- | 1.56 |
| | | | | | | quinoline | |
| ST1-76-3 | 12.5 | Nerolidol | 500 | Allyl | 1001 | HEDP | 1001 |
| | | | | isothiocyanate | | | |
| ST1-78-1 | 3.15 | Nerolidol | 500 | Usnic Acid | 6.25 | 2- | 1001 |
| | | | | | | hydroxypropyl- | |
| | | | | | | α- | |
| | | | | | | cyclodextrin | |
| ST2-8-2 | 0.39 | Limonene | 1001 | Na pyrithione | 0.79 | Salicylic acid | 1001 |
| ST1-72-1 | 1.56 | Lauryl | 12.5 | CPC | 3.15 | K2EDTA | 500 |
| | | Dimethylamine | | | | | |
| | | Oxide | | | | | |

| | | | | MIC- | MIC- | MIC- |
|---|---|---|---|---|---|---|
| | | other- | SI- | MRSA | MRSA | MRSA |
| | | MIC- | MRSA- | 2-way | 2-way | 2-way |
| Sample | Other | MRSA | other | 1 (1 + 2) | 2 (2 + 3) | 3 (1 + 3) |
| ST1-73-1 | Phenoxy- | 6080 | 0.251 | 1.56 | 50 | 1.56 |
| | ethanol | | | | | |
| ST1-76-3 | Phenoxy- | 6080 | 0.052 | 25 | 50 | 25 |
| | ethanol | | | | | |
| ST1-78-1 | Phenoxy- | 6080 | 0.514 | 25 | 25 | 50 |
| | ethanol | | | | | |
| ST2-8-2 | Phenoxy- | 6080 | 0.495 | 1.56 | 0.79 | 1001 |
| | ethanol | | | | | |
| ST1-72-1 | Phenoxy- | 6080 | 0.623 | 3.15 | 3.15 | 100 |
| | ethanol | | | | | |

What is claimed is:

1. An antimicrobial composition, comprising:
a synergistic combination of three or more agents, wherein each of the three or more agents is independently selected from the group of compounds consisting of sequestering agents, carbohydrates and carbohydrate derivatives, terpenes/terpenoids, amines and amine derivatives, plant-derived oils, sulfonates, phenols, fatty acids, dibenzofuran derivatives, organo isothiocyanates, quaternary ammonium compounds, peroxides and peroxide donors, and macrolide polyenes; and wherein at least two of the three or more agents are not selected from the same compound group.

2. The antimicrobial composition of claim 1, wherein at least one of the three or more agents is an antimicrobial agent, and wherein said antimicrobial agent is selected from the group of antimicrobial agents consisting of an antibacterial agent, an antifungal agent, and an antiviral agent;
wherein said antibacterial agent is selected from the group consisting of beta-lactams, aminoglycosides, glycopeptides, fluoroquinolones, macrolides, tetracyclines, and sulphonamides;
wherein said antifungal agent is selected from the group consisting of triazoles, imidazoles, polyene antimycotics, allylamines, echinocandins, cerulenin, and griseofulvin; and
wherein said antiviral agent is selected from the group consisting of reverse transcriptase inhibitors, nucleoside reverse transcriptase inhibitors (NRTIs), nucleoside analog reverse transcriptase inhibitors (NARTIs), guanine analogs, protease inhibitors, neuraminidase inhibitors, and nucleoside antimetabolites.

3. The antimicrobial composition of claim 2, wherein said beta-lactam is selected from the group consisting of penicillins, cephalosporins, carbapenems, and monobactams; and wherein said antiviral agent is selected from the group consisting of acyclovir, ribavarine, zidovudine, and idoxuridine.

4. The antimicrobial composition of claim 1, wherein at least one of the three or more agents is a sequestering agent, and the sequestering agent is selected from the group of sequestering agents consisting of quinolines, phosphorus acid derivatives, carboxylate sequestrants, natural protein sequestrants, and cyclodextric sequestrants.

5. The antimicrobial composition of claim 1, wherein at least one of the three or more agents is a sequestering agent, and the sequestering agent is selected from the group consisting of 8-hydroxyquinoline, 2-hydroxypropyl-α-cyclodextrin, di-potassium ethylenediaminetetraacetic acid (EDTA), tetra-sodium ethylenediaminetetraacetic acid (EDTA), 1-hydroxyethylidene-1,1-diphosphonic acid (HEDP), sodium pyrophosphate, potassium hypophosphite, sodium tripolyphosphate, salicylic acid, citric acid, lactoferrin, and hypophosphorous acid.

6. The antimicrobial composition of claim 1, wherein at least one of the three or more agents is a carbohydrate or carbohydrate derivative, and the carbohydrate is selected from the group consisting of polysaccharides, oligosaccharides and fatty acid polyol esters.

7. The antimicrobial composition of claim 1, wherein at least one of the three or more agents is a carbohydrate or carbohydrate derivative, and the carbohydrate or carbohydrate derivative is selected from the group consisting of chitosan, octyl glucoside, and glycerol monocaprylate.

8. The antimicrobial composition of claim 1, wherein at least one of the three or more agents is a terpene or terpenoid, and the terpene or terpenoid is selected from the group of terpenes/terpenoids consisting of limonene, nerolidol, linalool, totarol, and ursolic acid.

9. The antimicrobial composition of claim 1, wherein at least one of the three or more agents is an amine or amine derivative, and the amine or amine derivative is selected from the group of amine derivatives consisting of amines, amine oxides, peptides, alkaloids, and dyes having an amine functional group.

10. The antimicrobial composition of claim 1, wherein at least one of the three or more agents is an amine or amine derivative, and the amine or amine derivative is selected from the group consisting of N,N-bis(3-aminopropyl) dodecylamine, lauryl dimethylamine oxide, Na pyrithione, nisin, piperine, and methylene blue.

11. The antimicrobial composition of claim 1, wherein at least one of the three or more agents is a plant-derived oil, and the plant-derived oil is selected from the group consisting of allyl isothiocyanate and carvacrol.

12. The antimicrobial composition of claim 1, wherein at least one of the three or more agents is a sulfonate, and the sulfonate is selected from the group consisting of naphthalene sulfonic acid and sodium lignosulfonate.

13. The antimicrobial composition of claim 1, wherein at least one of the three or more agents is a phenol, and the phenol is selected from the group consisting of tannic acid, carvacrol, activin, octyl gallate, thymol, and catechins.

14. The antimicrobial composition of claim 1, wherein at least one of the three or more agents is a fatty acid, and the fatty acid is phospholipid CDM.

15. The antimicrobial composition of claim 1, wherein at least one of the three or more agents is a dibenzofuran derivative, and the dibenzofuran derivative is usnic acid.

16. The antimicrobial composition of claim 1, wherein at least one of the three or more agents is an organo isothiocyanate, and the organo isothiocyanate is allyl isothiocyanate.

17. The antimicrobial composition of claim 1, wherein at least one of the three or more agents is a quaternary ammonium compound, and the quaternary ammonium compound is selected from the group consisting of cetylpyridinium chloride, L-carnitine, and alkyl dimethyl benzyl ammonium chloride.

18. The antimicrobial composition of claim 1, wherein at least one of the three or more agents is a peroxide or peroxide donor, and the peroxide or peroxide donor is selected from the group consisting of hydrogen peroxide and sodium carbonate peroxyhydrate.

19. The antimicrobial composition of claim 1, wherein at least one of the three or more agents is a macrolide polyene, and the macrolide polyene is natamycin.

20. The antimicrobial composition of claim 1, further comprising a compound selected from the group consisting of phenoxyethanol, tetrahydrofurfuryl alcohol (THFA), block copolymers based on ethylene oxide and propylene oxide, polyethylene glycol, and water.

21. A method for treating a microbial infection, comprising administering an antimicrobial composition as an active ingredient, said antimicrobial composition comprising:
a synergistic combination of three or more agents, wherein each of the three or more agents is independently selected from the group of compounds consisting of sequestering agents, carbohydrates and carbohydrate derivatives, terpenes/terpenoids, amines and amine derivatives, plant-derived oils, sulfonates, phenols, fatty acids, dibenzofuran derivatives, organo isothiocyanates, quaternary ammonium compounds, peroxides and peroxide donors, and macrolide polyenes; and wherein at least two of the three or more agents are not selected from the same compound group.

22. The method of claim 21, wherein the microbial infection is a bacterial infection and wherein the bacterial infection is caused by a drug-resistant bacteria.

23. A method for treating a microorganism-contaminated surface, comprising applying to the surface an antimicrobial composition, said antimicrobial composition comprising:
a synergistic combination of three or more agents, wherein each of the three or more agents is independently selected from the group of compounds consisting of sequestering agents, carbohydrates and carbohydrate derivatives, terpenes/terpenoids, amines and amine derivatives, plant-derived oils, sulfonates, phenols, fatty acids, dibenzofuran derivatives, organo isothiocyanates, quaternary ammonium compounds, peroxides and peroxide donors, and macrolide polyenes; and wherein at least two of the three or more agents are not selected from the same compound group.

24. The method of claim 23, wherein the microorganisms of the microorganism-contaminated surface comprise drug-resistant bacteria.

25. The antimicrobial composition of claim 1, wherein the synergistic combination of three or more agents comprises an amine or amine derivative, carbohydrate or carbohydrate derivative, and a sequestering agent.

26. The antimicrobial composition of claim 25, wherein the synergistic combination of three or more agents comprises piperine, chitosan, and 1-hydroxyethylidene-1,1-diphosphonic acid (HEDP).

27. The antimicrobial composition of claim 1, wherein the synergistic combination of three or more agents comprises at least two agents each independently selected from the group consisting of amines and amine derivatives; and a sequestering agent.

28. The antimicrobial composition of claim 27, wherein the synergistic combination of three or more agents comprises piperine, methylene blue, and salicylic acid.

29. The antimicrobial composition of claim 1, wherein the synergistic combination of three or more agents comprises a terpene/terpenoid, an amine or amine derivative, and a sequestering agent.

30. The antimicrobial composition of claim 29, wherein the synergistic combination of three or more agents comprises nerolidol, nisin, and 2-hydroxypropyl-a-cyclodextrin.

31. The antimicrobial composition of claim 1, wherein the synergistic combination of three or more agents comprises two terpene/terpenoids and a sequestering agent.

32. The antimicrobial composition of claim 31, wherein the synergistic combination of three or more agents comprises nerolidol, limonene, and 1-hydroxyethylidene-1,1-diphosphonic acid (HEDP).

33. The antimicrobial composition of claim 1, wherein the synergistic combination of three or more agents comprises a terpene/terpenoid, a carbohydrate or carbohydrate derivative, and a sequestering agent.

34. The antimicrobial composition of claim 33, wherein the synergistic combination of three or more agents comprises nerolidol, octyl glucoside, and salicylic acid.

35. The antimicrobial composition of claim 1, wherein the synergistic combination of three or more agents comprises a terpene/terpenoid, a phenol, and a sequestering agent.

36. The antimicrobial composition of claim 35, wherein the synergistic combination of three or more agents comprises limonene, thymol, and citric acid.

37. The antimicrobial composition of claim 1, wherein the antimicrobial agents are selected from the group consisting of anti-tuberculosis drugs, antileprosy drugs, oxazolidelones, bisdiguanides, quaternary ammonium compounds, carbanilides, salicyanilides, hydroxydiphenyls, organometallic antiseptics, halogen antiseptics, peroxygens, amine derivatives, terpenes, terpenoids, phenols, alkaloids, natural alkyl isothiocyanates, organic sulfonates, fatty acid esters, and alkyl glycosides.

38. The antimicrobial composition of claim 1, wherein the antimicrobial agents are selected from the group consisting of hydantoins, 3-iodo-2-propynyl-butyl-carbamate (IPBC), isothiazolones, benzisothiazolones (BIT), chlorhexidine, 2,2-dibromo-3-nitrilo propionamide (DBNP), 2-bromo-2-nitropropane-1,3-diol, ureas, nisin, pyrithiones, N,N-bis(3-aminopropyl)dodecylamine, lauryl amine oxide, and cetylpyridinium chloride (CPC).

39. The antimicrobial composition of claim 1, wherein the synergistic combination of three or more agents comprises a terpene, an amine or amine derivative, and a phenol.

40. The antimicrobial composition of claim 39, wherein the synergistic combination of three or more agents comprises limonene, sodium pyrithione, and salicylic acid.

41. The antimicrobial composition of claim 1, wherein the synergistic combination of three or more agents comprises an amine or amine derivative, a quaternary ammonium compound, and a sequestering agent.

42. The antimicrobial composition of claim 41, wherein the synergistic combination of three or more agents comprises lauryl amine oxide, cetylpyridinium chloride (CPC), and potassium ethylenediaminetetraacetic acid.
